SEQUENCE LISTING (16 sequences)

SEQ ID NO: 1 - protein, 22 aa, synthetic construct
PVLDLFRELLNELLEALKQKLK

SEQ ID NO: 2 - protein, 25 aa, synthetic construct
PVLDLFRELLNELLEALKQKLKGGC

SEQ ID NO: 3 - protein, 25 aa, synthetic construct
CGGPVLDLFRELLNELLEALKQKLK

SEQ ID NO: 4 - protein, 24 aa, synthetic construct
CPVLDLFRELLNELLEALKQKLKC

SEQ ID NO: 5 - DNA, 16 bp, synthetic construct
  source 1..16: mol_type = other DNA
tggcaagcatcctgta

SEQ ID NO: 6 - DNA, 21 bp, synthetic construct
  source 1..21: mol_type = other DNA
gcgtttgctcttcttcttgcg

SEQ ID NO: 7 - DNA, 26 bp, synthetic construct
  source 1..26: mol_type = other DNA
cggaatcgtgaatgcttatacatccg

SEQ ID NO: 8 - DNA, 20 bp, synthetic construct
  source 1..20: mol_type = other DNA
gcctcagtctgcttcgcacc

SEQ ID NO: 9 - DNA, 15 bp, synthetic construct
  source 1..15: mol_type = other DNA
ggttggattggttgg

SEQ ID NO: 10 - DNA, 15 bp, synthetic construct
  source 1..15: mol_type = other DNA
ggttggatcggttgg

SEQ ID NO: 11 - DNA, 30 bp, synthetic construct
  source 1..30: mol_type = other DNA
ctccaacatcaaggaagatggcatttctag

SEQ ID NO: 12 - DNA, 18 bp, synthetic construct
  source 1..18: mol_type = other DNA
tcactttcataatgctgg

SEQ ID NO: 13 - DNA, 20 bp, synthetic construct
  source 1..20: mol_type = other DNA
tcttggttacatgaaatccc

SEQ ID NO: 14 - DNA, 25 bp, synthetic construct
  source 1..25: mol_type = other DNA
gttgcctccggttctgaaggtgttc

SEQ ID NO: 15 - DNA, 21 bp, synthetic construct
  source 1..21: mol_type = other DNA
cctccggttctgaaggtgttc

SEQ ID NO: 16 - DNA, 22 bp, synthetic construct
  source 1..22: mol_type = other DNA
caatgccatcctggagttcctg

(19) United States
(12) Patent Application Publication
(10) Pub. No.: US 2025/0339539 A1
(43) Pub. Date: Nov. 6, 2025

(54) Phosphate Membrane Nanodiscs Conjugated to Therapeutic Agents and Medical Uses Thereof

(71) Applicant: Emory University, Atlanta, GA (US)

(72) Inventors: Khalid Salaita, Atlanta, GA (US); Radhika Sharma, Atlanta, GA (US)

(21) Appl. No.: 18/730,610

(22) PCT Filed: Jan. 20, 2023
(86) PCT No.: PCT/US2023/060984

§ 371 Date: Jul. 19, 2024

Related U.S. Application Data
(60) Provisional application No. 63/301,284, filed on Jan. 20, 2022.

(51) Int. Cl. A61K 47/54; A61K 47/64; A61K 47/69; A61P 35/00

(52) U.S. Cl. CPC A61K 47/544; A61K 47/64; A61K 47/6929; A61P 35/00

(57) ABSTRACT

This disclosure relates to phosphate membrane nanodiscs covalently modified with therapeutic agents such as antisense oligonucleotides or other nucleobase polymers and medical uses related thereto. In certain embodiments, the phosphate membrane nanodiscs comprise a phospholipid having a thiol group used for conjugation to agents such as oligonucleotides or other nucleobases polymers having a thiol reactive group. In certain embodiment, the phosphate membrane nanodiscs comprise a stabilizing peptide having a thiol group used for further conjugation to therapeutic agents.

### CROSS-REFERENCE TO RELATED APPLICATIONS

This application claims the benefit of U.S. Provisional Application No. 63/301,284 filed Jan. 20, 2022. The entirety of this application is hereby incorporated by reference for all purposes.

### STATEMENT REGARDING FEDERALLY SPONSORED RESEARCH OR DEVELOPMENT

This invention was made with government support under HL142866 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 22074PCT.xml. The XML file is 15 KB, was created on Jan. 19, 2023, and is being submitted electronically via the USPTO patent electronic filing system.

### BACKGROUND

Clinical trials using nucleic acid-based therapeutics sometime fail because these agents are unable to sufficiently reach their cytoplasmic target, which may be due to biological processes such as nuclease-mediated degradation and endosomal entrapment. Using larger doses of nucleic acid drugs can result in undesirable off-target effects and adverse drug reactions. Thus, there is a need to identify improvements.

Lipid-based nanodiscs contain a phosphate lipid membrane and have emerged as a class of nanoparticles for the delivery of nucleic acids. These phosphate membrane nanodiscs structurally mimic nascent high-density lipoproteins (HDL) that circulate in blood, which function in reverse cholesterol transport, and are primarily comprised of phospholipids along with apolipoprotein A1 (ApoA1), an alpha-helical scaffolding protein.

Vickers et al. report microRNAs are transported in plasma and delivered to recipient cells by high-density lipoproteins (HDLs). Nat Cell Biol, 2011, 13(4):423-433.

Rad et al. report a universal discoidal nanoplatform for the intracellular delivery of peptide nucleic acids. Nanoscale, 2019, 11, 12517.

Fawaz et al. report phospholipid component defines pharmacokinetic and pharmacodynamic properties of synthetic high-density lipoproteins. J Pharmacol Exp Ther, 2020, 372:193-204.

Tenchov et al. report lipid nanoparticles (LNPs) as promising vehicles to deliver oligonucleotide therapeutics. ACS Nano, 2021, 15, 16982-17015. See also U.S. Pat. No. 8,734,853 and US Patent Application Publication No. 2018/0250419.

References cited herein are not an admission of prior art.

### SUMMARY

This disclosure relates to phosphate membrane nanodiscs covalently modified with therapeutic agents such as antisense oligonucleotides or other nucleobase polymers and medical uses related thereto. In certain embodiments, the phosphate membrane nanodiscs comprise a phospholipid having a thiol group used for conjugation to agents such as oligonucleotides or other nucleobases polymers having a thiol reactive group. In certain embodiment, the phosphate membrane nanodiscs comprise a stabilizing peptide having a thiol group used for further conjugation to therapeutic agents.

In certain embodiments, this disclosure relates to phosphate membrane nanodiscs covalently conjugated to a therapeutic agent, wherein the phosphate membrane nanodiscs comprises a zwitterionic phospholipid, a phospholipid having a thiol group, a nanodisc stabilizing peptide, such as an ApoA1, variant, or fragment thereof, and wherein the therapeutic agent is conjugated to the phospholipid having a thiol group providing a thiol-linked adduct.

In certain embodiments, this disclosure relates to phosphate membrane nanodiscs covalently conjugated to a therapeutic agent, wherein the phosphate membrane nanodiscs comprise a zwitterionic phospholipid, a phospholipid having a thiol group, a nanodisc stabilizing peptide, such as an ApoA1, variant, or fragment thereof, comprising a C-terminal thiol group, C-terminal cysteine amino acid, a GC sequence, or GGC sequence, wherein the therapeutic agent is conjugated to a phospholipid providing a thiol-linked adduct; and wherein the therapeutic agent is conjugated to the stabilizing peptide providing a thiol-linked adduct to the C-terminal thiol group, C-terminal cysteine amino acid, a GC sequence, or GGC sequence. In certain embodiments, the therapeutic agent is a nucleobase polymer and the thiol-linked adduct is a thiol-maleimide adduct.

In certain embodiments, the phosphate membrane nanodiscs are conjugated with 12, 13, 14, or 15 greater therapeutic agents, e.g., nucleobase polymers, antisense oligonucleotide, on each individual phosphate membrane nanodisc, wherein the nanodisc has an average diameter of about between 8 to 17 nm, or between 10 to 20 nm.

In certain embodiments, the phosphate membrane nanodiscs are made by the process of contacting the zwitterionic phospholipid, a phospholipid having a thiol group, and a nanodisc stabilizing peptide, wherein the molar ratio or weight ratio of the zwitterionic phospholipid and the phospholipid having a thiol group is between 95 to 5 and 90 to 10 respectively.

In certain embodiments, the phosphate membrane nanodiscs is made by the process of contacting the zwitterionic phospholipid, a phospholipid having a thiol group, and a nanodisc stabilizing peptide, at a temperature between 40 to 55 degrees Celsius or between 35 to 60 degrees Celsius.

In certain embodiments, the phosphate membrane nanodiscs is made by the process of contacting the zwitterionic phospholipid, a phospholipid having a thiol group, and a nanodisc stabilizing peptide, in an aqueous solution at a pH between 7.5 to 8.5 or between 7.0 to 9.0.

In certain embodiments, the zwitterionic phospholipid is 1,2-dimyristoyl-sn-glycero-3-phosphocholine (DMPC).

In certain embodiments, the phospholipid having a thiol group is 1,2-dipalmitoyl-sn-glycero-3-phosphothioethanol.

In certain embodiments, the molar ratio or weight ratio of the zwitterionic phospholipid to the phospholipid having a thiol group is between 8:1 to 10:1 or between 8:1 to 20:1.

In certain embodiments, the nanodisc stabilizing peptide comprises a C-terminal thiol group, optionally conjugated to the peptide by a linking group, cysteine amino acid, a GC sequence, or GGC sequence.

In certain embodiments, the phosphate membrane nanodiscs comprise a cationic and/or zwitterionic phospholipid, a phospholipid having a thiol group, a stabilizing peptide, and a therapeutic agent, such as an anti-sense oligonucleotide (ASO) or other nucleobase polymer. In certain embodiments, the cationic and/or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in a molecular ratio of 9:1 or between 8:1 and 10:1. In certain embodiments, the stabilizing peptide sequence consists of or comprises PVLDLFRELLNELLEALKQKLK (SEQ ID NO: 1) or PVLDLFRELLNELLEALKQKLKGGC (SEQ ID NO: 2).

In certain embodiments, this disclosure relates to methods of treating diseases or conditions comprising administering to a subject in need thereof an effective amount of a phosphate membrane nanodisc as disclosed herein comprising a therapeutic agent/oligonucleotide that can treat the disease or conditions, e.g., a therapeutic agent can specifically bind/degrade/inhibit a disease or condition associated biomolecule.

In certain embodiments, this disclosure relates to methods of treating cancer comprising administering to a subject in need thereof an effective amount of a phosphate membrane nanodisc as disclosed herein optionally in combination with another anticancer agent. In certain embodiments, the phosphate membrane nanodisc comprises or is coated with a nucleobase polymer or antisense oligonucleotide that specifically binds HIF-1-alpha mRNA and/or induces RNase H cleavage. In certain embodiments, the nucleobase polymer or antisense oligonucleotide comprises TGGCAAGCATCCTGTA (SEQ ID NO: 5). In certain embodiments, this disclosure relates to phosphate membrane nanodiscs wherein the nanodisc stabilizing peptide comprises the amino acid sequence of PVLDLFRELLNELLEALKQKLKGGC (SEQ ID NO: 2) and the therapeutic agent is an antisense oligonucleotide comprising the sequence TGGCAAGCATCCTGTA (SEQ ID NO: 5).

In certain embodiments, the cancer is pancreatic cancer, liver cancer, kidney cancer, lung cancer, non-small cell lung cancer, or small cell lung cancer. In certain embodiments, the cancer is breast cancer, lung cancer, bronchus cancer, prostate cancer, colon cancer, rectum cancer, melanoma of the skin, bladder cancer, lymphoma, kidney cancer, renal cancer, pelvis cancer, endometrial cancer, leukemia, pancreatic cancer, thyroid cancer, or liver cancer.

In certain embodiments, this disclosure relates to pharmaceutical compositions and kits comprising phosphate membrane nanodisc as reported herein. In certain embodiments, this disclosure relates to the production of a medicament comprising phosphate membrane nanodisc as reported herein for therapeutic uses reported herein.

### BRIEF DESCRIPTION OF THE SEVERAL VIEWS OF THE DRAWINGS

FIG. 1A illustrates a protocol for preparing and assembling DNA-phosphate membrane nanodisc conjugates. Phosphate membrane nanodiscs (NDs) are formed by preparing 80 nm unilamellar vesicles (SUVs) primarily using DMPC as a major component and the thiol phospholipid as a minor component (about 10%). A peptide ApoA1mimetic is added to SUVs before subjecting them to thermal cycling between 55° C. and 4° C. to form NDs.

FIG. 1B illustrates DNA bearing a maleimide group is chemically conjugated to the thiol NDs resulting in the DNA-ND conjugate. The product is purified by using size exclusion chromatography.

FIG. 1C shows data from experiments on coupling and optimizing DNA onto the ND surface. TEM images and binned size analysis of NDs composed of DMPC only, 5% thiolated lipid, 10% thiolated lipid, and after DNA is coupled onto a 10% thiol ND surface. Samples were prepared using a plasmon-etched 400-mesh copper grid, and staining was performed using Nano-W. Left, shows a plot comparing DNA density of DNA-ND conjugates consisting of NDs with 5% thiol or 10% thiol. The 10% thiol ND shows a greater DNA density (3 DNA/ND vs 1.6 DNA/ND for 5%) at standard reaction conditions: 25° C., pH 7.4. Right shows a plot comparing the DNA density on 10% thiol ND after testing different pH (7.5 and 8.5) and temperature (25° C. and 45° C.) conditions. There is an average increase in DNA density from 3±0.5 DNA/ND to 13±2 DNA/ND under the improved conditions.

FIGS. 2A-2B show data on validating and characterizing the attachment of DNA onto the surface of NDs. DLS graphs indicate a shift in the hydrodynamic radius from 13.0 to 16.0 nm after conjugating DNA to the NDs. Size distribution profile is a representative graph of a typical sample (n=3 independent experiments) containing NDs or NDs with DNA conjugate.

FIG. 2A shows a zeta-potential graph showing the increase in negative charge from −10.9 to −35.2 mV after DNA is coupled onto the surface of the NDs.

FIG. 2B shows a schematic representation the different samples: DNA only, ND only, DNA-ND, and DNA mixed with NDs, that were used for the FRET assay. TYE labeled DNA and Cy5 labeled phospholipid NDs were used for FRET. Fluorescence spectra of the four groups were excited at λ=520 nm. DNA was measured at a concentration of 100 nM, while 8 nM ND concentration was used to best match the ND and DNA plus ND mix concentration in the DNA-ND sample. Plot of the calculated FRET efficiency of the chemically conjugated DNA-NDs compared to the unlinked DNA and ND mixture. The higher FRET efficiency (40.1%) of the DNA-ND compared to the mixed control (4.5%) further substantiates that DNA is bound onto the surface of the ND.

FIG. 3 shows data indicating HeLa, U373, and PLC/PRF/5 cells uptake ASO-NDs in a dose- and time-dependent manner. Confocal microscopy images were obtained on a time course for the uptake of 100 nM ASO-ND into HeLa cells. ASO was labeled with a TYE dye and the NDs were labeled with a Cy5 phospholipid. Cells were fixed and stained with DAPI before acquiring images. Pearson's colocalization coefficient analysis of TYE and Cy5 signal in n=25-30 cells for each group. Analysis indicates the increased detachment of ASO from the ND within 12 h. Flow cytometry histograms were evaluated at 12 h for HeLa, U373, and PLC/PRF/5 cells that were treated with the ASO-ND and rinse prior to the measurement. Flow data represents intensities for a minimum of 5000 cells. Increase in uptake of ASO-ND over time was measured at 4, 12, and 24 h as measured from the mean intensity from flow cytometry in HeLa, U373, and PLC/PRF/5 cells.

FIGS. 4A and 4B show data indicating SRB1 partially mediates uptake of ASO-NDs in HeLa, U373, and PLC/PRF/5 cells.

FIG. 4A shows a schematic indicating the blocking of SRB1 on the cell surface with BLT-1 hindering the internalization of ASO-ND. Flow cytometry histograms were used to measure Cy5 intensity for cells that were pretreated with 50 μM BLT-1 for 1 h, and then incubated with the ASO-ND for 2 h in HeLa, U373, and PLC/PRF/5 cells.

FIG. 4B shows data comparing the uptake of ASO-ND or ND into cells after BLT-1 treatment. The values are normalized to the uptake level measured for the untreated control group.

FIGS. 5A-5G show data quantifying the functional activity of ASO-NDs and ASOs that target HIF-1-alpha in three model cell lines.

FIG. 5A shows a schematic of the HIF-1-alpha transcript where the poly-A tail was denoted with circles, a target region, and a 5′ capping is show with a circle.

FIG. 5B shows a plot quantifying the uptake of ASO-ND and ASO in Hela cells treated for 24 h as a function of ASO concentration. The ASO was tagged with a TYE dye and the mean fluorescence intensity per cell was determined using flow cytometry.

FIG. 5C shows a plot comparing HIF-1-alpha levels in Hela cells treated for 24 h with ASO and ASO-ND. Quantification was performed using RT-qPCR. Values were normalized to untreated control cells.

FIG. 5D shows a plot of HIF-1-alpha levels determined by using RT-qPCR for HeLa cells that were treated with 75 nM concentrations of ASO-ND, scrambled-ND, ND, and ASO for 24 h. The ASO concentration was matched at 75 nM; however, the ND group used a 7 nM concentration of ND. The transcript levels are normalized to the untreated control group.

FIG. 5E show data on U373 cells.

FIG. 5F shows data on PLC/PRF/5 cells.

FIG. 5G shows data from cell viability assessments in HeLa cells after dosing the HeLa cells with ASO-ND conjugates, ASO Only, Scrambled-ND, and ND only. Cell viability was assessed using MTT assay. HeLa cells were subjected to 75 nM ASO, and the ND group was subjected to 7 nM ND to best match ND concentration from the ASO-ND groups. The cells were incubated with sample for either 24 or 48 h before adding MTT reagent and performing the assay. The values are normalized to the OD measured at 590 nm for the untreated cells as a control.

FIG. 6A illustrates the assembly and synthesis of phosphate membrane nanodiscs conjugated to nucleic acids including through the stabilizing ApoA1peptide-nanodiscoidal nucleic acids (NNA). The ND scaffold is assembled by preparing 80 nm small unilamellar vesicles (SUVs) and combining them with a modified ApoA1mimetic peptide containing a Cys (C) amino acid insertion. The NNA is generated by conjugating maleimide-linked DNA to the exposed thiols on the surface (lipid) and edge (peptide) of the scaffold.

FIG. 6B shows a table of lipids used in the assembly of NDs and NNAs. DMPC is the majority component present in the NDs and the thiol lipid, Ptd-Thioethanol, is added to certain discs to prepare NNAs.

FIG. 6C shows a table of ApoA1mimetic peptides screened for the formation of NNAs. The original mimetic peptide, denoted as A (SEQ ID NO: 1), does not contain any Cys (C) residues and was further modified in versions B-D at the N- and/or C-terminus (SEQ ID NOs: 2-4).

FIG. 6D shows a panel of ND scaffolds generated from the peptide screen. Excluding peptide A, each peptide was used to prepare two different versions of DNA conjugates, one with DMPC exclusively and referred to as NDs, and a second type which included thiolated phospholipids and denoted as the nanodiscoidal nucleic acids (NNA).

FIG. 7 shows data quantifying the functional activity of ASO-NDs and NNAs for reducing HIF-1-alpha mRNA levels in different cell lines. Plot of HIF-1-alpha levels after incubating HeLa cells with ASO-NDs (NDs 1-2, 4) and NNAs (NDs 3 and 5) for 24 h prior to extracting mRNA and quantifying the levels via RT-qPCR. The anti-HIF-1-alpha ASO (EZN2968) was used. The ASO concentration was 100 nM for all groups, including the scrambled (scr.) ASO (conjugated on a ND) and ASO only. The ASO-ND and NNA conjugated to the peptide on the C-terminus (NDs 2-3) had greater activity compared to the N-terminus (NDs 4-5). Assessment of cell viability using MTT assay. HeLa cells were subject to 100 nM of ASO-ND or NNA and incubated for 24 h prior to adding MTT reagent and performing the assay. The values are normalized to the OD measured at 590 nm for the untreated cells as a control (ctrl). Cells treated with ASO-ND or NNAs demonstrated a significant reduction in viability, further confirming the functional activity of the conjugates. Each data point represents the percent viability for one replicate (n=4 independent replicates). Quantification of HIF-1-alpha levels as determined via RT-qPCR in KPC, LX-2 Human Hepatic Stellate, and HepG2 cells after incubating cells with 100 nM of NNA (ND 3), scrambled (scr.) ASO on a ND, and ASO only for 24 h. Transcript levels are normalized to the untreated control group (ctrl). The NNA scaffold significantly boosted ASO activity compared to cells administered 100 nM of ASO only without a scaffold.

FIG. 8 shows data on the NNA uptake and functional activity in H1299 3D spheroids. Confocal images of internalized NNAs and ASO (EZN2968) were obtained and visualized inside the spheroid cross section at the middle slice after 24 h of incubation (ASO concentration=100 nM) including brightfield (BF) image and fluorescence intensity of TYE563 (ASO) and Cy5 (NNA). Radial profile scan averaging TYE563 fluorescence intensity and normalizing for 14 spheroids (n=3 independent replicates). The profile scan reveals that the NNA uptake is more enhanced for spheroids, with the ability to penetrate the hypoxic core within 24 h. Mean TYE563 fluorescence quantification on the uptake of ASO and NNA (ND 3) as measured via flow cytometry. Flow cytometry histogram provided a difference in uptake of NNAs into spheroids after blocking SRB1 with 50 μm of BLT-1 for 1 h. NNAs (150 nM ASO and 6 nM ND) were subsequently incubated with spheroids for 2 h prior to dissociating the spheroids into individual cells and measuring cell associated fluorescence using flow cytometry. Mean Cy5 fluorescence quantification for the uptake of NNA (3) into spheroids treated with BLT-1. There is a 54% reduction in the uptake of NNAs after BLT-1 treatment. Plot comparing the HIF-1-alpha levels as measured by RTqPCR after subjecting the spheroids to 550 nM of ASO, ASO on NNA, or scrambled (scr.) ASO on a ND for 24 h. Transcript levels are normalized to the untreated control group (ctrl).

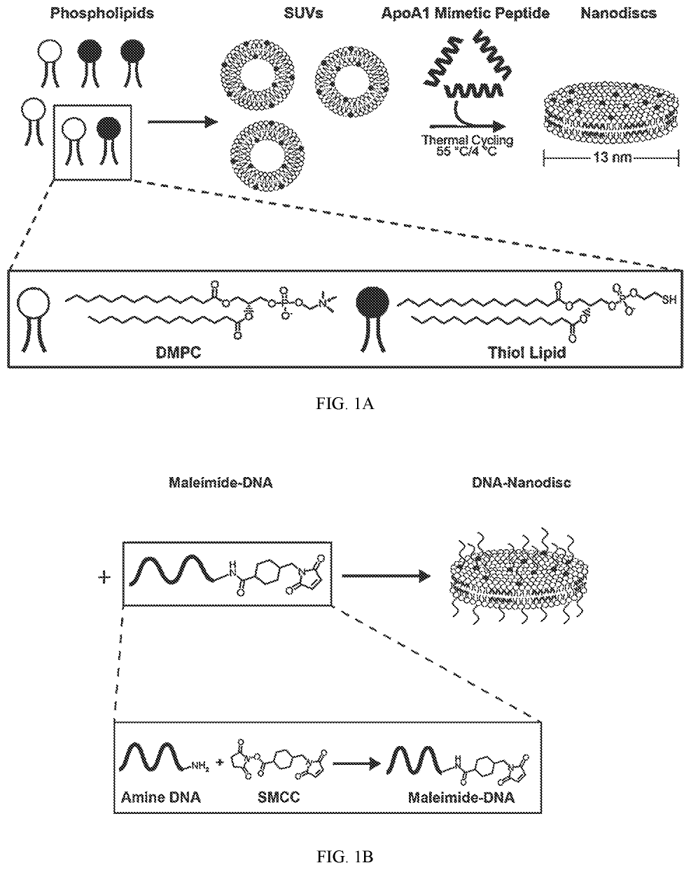

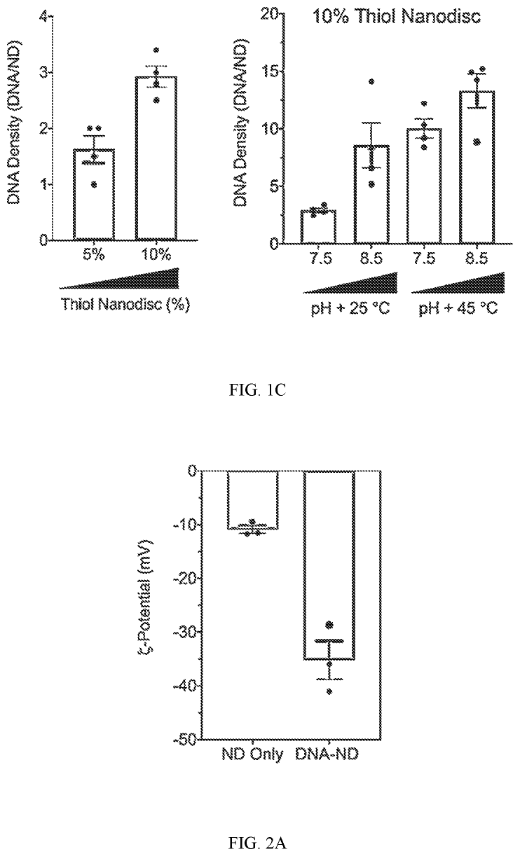

### DETAILED DESCRIPTION

Before the present disclosure is described in greater detail, it is to be understood that this disclosure is not limited to embodiments described, and as such may, of course, vary. An “embodiment” refers to an example and is not necessarily limited to such example. It is also to be understood that the terminology used herein is for describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps. The term “comprising” in reference to an oligonucleotide having a nucleic acid sequence refers to an oligonucleotide or peptide that may contain additional 5′ (5′ terminal end) or 3′ (3′ terminal end) nucleotides or N- or C-terminal amino acids, i.e., the term is intended to include the oligonucleotide sequence or peptide sequence within a larger nucleic acid or peptide.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein. The term “consisting of” in reference to an oligonucleotide or peptide having a nucleotide or peptide sequence refers an oligonucleotide or peptide having the exact number of nucleotides or amino acids in the sequence and not more or having not more than a range of nucleotides expressly specified in the claim. For example, “5′ sequence consisting of” is limited only to the 5′ end, i.e., the 3′ end may contain additional nucleotides. Similarly, a “3′ sequence consisting of” is limited only to the 3′ end, and the 5′ end may contain additional nucleotides.

The term “conjugated” refers to linking molecular entities through covalent bonds, or by other specific binding interactions, such as due to hydrogen bonding or other van der Walls forces. The force to break a covalent bond is high, e.g., about 1500 pN for a carbon-to-carbon bond. The force to break a combination of strong protein interactions is typically a magnitude less, e.g., biotin to streptavidin is about 150 pN. Thus, a skilled artisan would understand that conjugation must be strong enough to restrict the breaking of bonds in order to implement the intended results. In certain embodiments, the term conjugated is intended to include linking molecular entities that do not break unless exposed to a force of about greater than about 5, 10, 25, 50, 75, 100, 125, or 150 pN depending on the context.

A “linking group” refers to any variety of molecular arrangements that can be used to bridge or conjugate molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)—, —C(Hal) H—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRₙ—, —(C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH═CH₂, —CCH, —OH, —SH, —NH₂, —N₃, —CN, or —Hal, or two branched Rs may form an aromatic or non-aromatic cyclic structure. It is contemplated that in certain instances, the total Rs or “n” may be less than 100 or 50 or 25 or 10. Examples of linking groups include bridging alkyl groups, alkoxyalkyl, polyethylene glycols, amides, esters, and aromatic groups.

The terms, “nucleic acid,” or “oligonucleotide,” is meant to include nucleic acids, ribonucleic or deoxyribonucleic acid, mixtures, nucleobase polymers, or analog thereof. An oligonucleotide can include native or non-native bases. In this regard, a native deoxyribonucleic acid can have one or more bases selected from the group consisting of adenine, thymine, cytosine or guanine and a ribonucleic acid can have one or more bases selected from the group consisting of uracil, adenine, cytosine, or guanine.

The term “nucleobase polymer” refers to nucleic acids and chemically modified forms with nucleobase monomers. In certain embodiments, methods and compositions disclosed herein may be implemented with nucleobase polymers comprising units of a ribose, 2′deoxyribose, locked nucleic acids (1-(hydroxymethyl)-2,5-dioxabicyclo[2.2.1]heptan-7-ol), 2′-O-methyl groups, a 3′-3′-inverted thymidine, phosphorothioate linkages, or combinations thereof. In certain embodiments, the nucleobase polymer may be less than 100, 50, or 35 nucleotides or nucleobases.

Nucleobase monomers are nitrogen containing aromatic or heterocyclic bases that bind to naturally occurring nucleic acids through hydrogen bonding otherwise known as base pairing. A typical nucleobase polymer is a nucleic acid, RNA, DNA, or chemically modified form thereof. A nucleobase polymer may be single or double stranded or both, e.g., they may contain overhangs. Nucleobase polymers may contain naturally occurring or synthetically modified bases and backbones. In certain embodiments, a nucleobase polymer need not be entirely complementary, e.g., may contain one or more insertions, deletions, or be in a hairpin structure provided that there is sufficient selective binding.

With regard to the nucleobases, it is contemplated that the term encompasses isobases, otherwise known as modified bases, e.g., are isoelectronic or have other substitutes configured to mimic naturally occurring hydrogen bonding base-pairs, e.g., within any of the sequences herein U may be substituted for T, or T may be substituted for U. Examples of nucleotides with modified adenosine or guanosine include, but are not limited to, hypoxanthine, xanthine, 7-methylguanine. Examples of nucleotides with modified cytidine, thymidine, or uridine include 5,6-dihydrouracil, 5-methylcytosine, 5-hydroxymethylcytosine. Contemplated isobases include 2′-deoxy-5-methylisocytidine (iC) and 2′-deoxy-isoguanosine (iG) (see U.S. Pat. Nos. 6,001,983; 6,037,120; 6,617,106; and 6,977,161).

Nucleobase polymers may be chemically modified, e.g., within the sugar backbone or on the 5′ or 3′ ends. As such, in certain embodiments, nucleobase polymers disclosed herein may contain monomers of phosphodiester, phosphorothioate, methylphosphonate, phosphorodiamidate, piperazine phosphorodiamidate, ribose, 2′-O-methy ribose, 2′-O-methoxyethyl ribose, 2′-fluororibose, deoxyribose, 1-(hydroxymethyl)-2,5-dioxabicyclo[2.2.1]heptan-7-ol, P-(2-(hydroxymethyl)morpholino)-N,N-dimethylphosphon amidate, morpholin-2-ylmethanol, (2-(hydroxymethyl)morpholino)(piperazin-1-yl)phosphinate, or peptide nucleic acids or combinations thereof.

In certain embodiments, the nucleotide base polymer is single or double stranded and/or is 5′ end polyphosphorylated, e.g., di-phosphate, tri-phosphate and/or 3′ end capped with one, two, or more thymidine nucleotides. In certain embodiments, the nucleobase polymer can be modified to contain a phosphodiester bond, methylphosphonate bond or phosphorothioate bond. The nucleobase polymers can be modified, for example, 2′-amino, 2′-C-allyl, 2′-fluoro, 2′-O-methyl, 2′-H of the ribose ring. Constructs can be purified by gel electrophoresis using general methods or can be purified by high pressure liquid chromatography and re-suspended in water.

In certain embodiments, nucleobase polymers include one or more (e.g., about 1, 2, 3, 4, 5, 6, 7, 8, 9, 10, or more) LNA “locked nucleic acid” nucleotides such as a 2′,4′-C methylene bicyclo nucleotide (see for example U.S. Pat. Nos. 6,639,059, 6,670,461, 7,053,207). In certain embodiments, the disclosure features modified nucleobase polymers, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

As used herein, “subject” refers to any animal, preferably a human patient, livestock, or domestic pet.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

The term “effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular agent chosen, the dosing regimen to be followed, whether it is administered in combination with other agents, timing of administration, the tissue to which it is administered, and the physical delivery system in which it is carried.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation of the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

The cancer to be treated in the context of the present disclosure may be any type of cancer or tumor. These tumors or cancer include, and are not limited to, tumors of the hematopoietic and lymphoid tissues or hematopoietic and lymphoid malignancies, tumors that affect the blood, bone marrow, lymph, and lymphatic system. Hematological malignancies may derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin.

Also contemplated are malignancies located in the lung, colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax and, more particularly, childhood acute lymphoblastic leukemia, acute lymphoblastic leukemia, acute lymphocytic leukemia, acute myeloid leukemia, adrenocortical carcinoma, adult (primary) hepatocellular cancer, adult (primary) liver cancer, adult acute lymphocytic leukemia, adult acute myeloid leukemia, adult Hodgkin's disease, adult Hodgkin's lymphoma, adult lymphocytic leukemia, adult non-Hodgkin's lymphoma, adult primary liver cancer, adult soft tissue sarcoma, AIDS-related lymphoma, AIDS-related malignant tumors, anal cancer, astrocytoma, cancer of the biliary tract, cancer of the bladder, bone cancer, brain stem glioma, brain tumors, breast cancer, cancer of the renal pelvis and ureter, primary central nervous system lymphoma, central nervous system lymphoma, cerebellar astrocytoma, brain astrocytoma, cancer of the cervix, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood brain astrocytoma, childhood extracranial germ cell tumors, childhood Hodgkin's disease, childhood Hodgkin's lymphoma, childhood visual pathway and hypothalamic glioma, childhood lymphoblastic leukemia, childhood medulloblastoma, childhood non-Hodgkin's lymphoma, childhood supratentorial primitive neuroectodermal and pineal tumors, childhood primary liver cancer, childhood rhabdomyosarcoma, childhood soft tissue sarcoma, childhood visual pathway and hypothalamic glioma, chronic lymphocytic leukemia, chronic myeloid leukemia, cancer of the colon, cutaneous T-cell lymphoma, endocrine pancreatic islet cells carcinoma, endometrial cancer, ependymoma, epithelial cancer, Ewing's sarcoma and related tumors, cancer of the exocrine pancreas, extracranial germ cell tumor, extragonadal germ cell tumor, extrahepatic biliary tract cancer, cancer of the eye, breast cancer in women, Gaucher's disease, cancer of the gallbladder, gastric cancer, gastrointestinal carcinoid tumor, gastrointestinal tumors, germ cell tumors, gestational trophoblastic tumor, head and neck cancer, hepatocellular cancer, Hodgkin's disease, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, cancer of kidney, cancer of the larynx, cancer of the lip and mouth, cancer of the liver, cancer of the lung, lymphoproliferative disorders, macroglobulinemia, breast cancer in men, malignant mesothelioma, malignant thymoma, medulloblastoma, melanoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-Hodgkin's lymphoma during pregnancy, non-melanoma skin cancer, non-small cell lung cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, pancreatic cancer, paraproteinemias, purpura, parathyroid cancer, cancer of the penis, phaeochromocytoma, hypophysis tumor, neoplasia of plasmatic cells/multiple myeloma, primary central nervous system lymphoma, primary liver cancer, prostate cancer, rectal cancer, renal cell cancer, cancer of the renal pelvis and ureter, retinoblastoma, rhabdomyosarcoma, cancer of the salivary glands, sarcoidosis, sarcomas, skin cancer, small cell lung cancer, small intestine cancer, soft tissue sarcoma, squamous neck cancer, stomach cancer, pineal and supratentorial primitive neuroectodermal tumors, T-cell lymphoma, testicular cancer, thymoma, thyroid cancer, transitional cell cancer of the renal pelvis and ureter, transitional renal pelvis and ureter cancer, trophoblastic tumors, cell cancer of the renal pelvis and ureter, cancer of the urethra, cancer of the uterus, uterine sarcoma, vaginal cancer, optic pathway and hypothalamic glioma, cancer of the vulva, Waldenstrom's macroglobulinemia, Wilms' tumor and any other hyperproliferative disease, as well as neoplasia, located in the system of a previously mentioned organ.

A “chemotherapy agent,” “chemotherapeutic,” “anti-cancer agent,” or the like, refer to molecules that are recognized to aid in the treatment of a cancer. Contemplated examples include the following molecules or derivatives such as abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC). In certain embodiments, the chemotherapy agent is an anti-PD-1, anti-PD-L1 anti-CTLA4 antibody or combinations thereof, such as an anti-CTLA4 (e.g., ipilimumab, tremelimumab) and anti-PD1 (e.g., nivolumab, pembrolizumab, cemiplimab) and anti-PD-L1 (e.g., atezolizumab, avelumab, durvalumab).

### Phosphate Membrane Nanodiscs

Nanodiscs containing phospholipid bilayer like membranes can be generated using stabilizing scaffold proteins or synthetic polymers. Typically, the stabilizing protein, or functional variant, is a form of a natural Apolipoprotein A-I, e.g., ApoA1, (truncated or operable variants) which forms a complex with the phospholipid components. Hydrophobic and hydrophilic interactions between the stabilizing protein and phospholipids creates disc like shapes that are typically water soluble and mimic a cell-membrane environment. Often the diameter of the phosphate membrane nanodisc ranges from about 5 to 20 nm which can vary depending on the apolipoprotein length and sequence.

Disclosed herein are methods to boost nucleic acid density on a phosphate membrane nanodisc scaffold by doping in thiol-containing phospholipids into the phosphate membrane nanodiscs and conjugating these lipids to maleimide-modified therapeutics or oligonucleotides forming covalent linkages. This conjugation chemistry offered significant advantages over commonly used non-covalent modifications of spherical and other discoidal HDL scaffolds (including HPPS, sHDL, and NDs). Specifically, non-covalent interactions employing electrostatic binding and cholesterol-mediated binding are weak, yield, low nucleic acid density, high polydispersity of loading, and display short half-lives (2-4 h) in vitro and in vivo, thus limiting translational potential. ND scaffolds reported herein are provided to maximize loading density on the ND scaffold, also referred to as “phosphate membrane nanodiscs.”

This disclosure relates to phosphate membrane nanodiscs covalently modified with therapeutic agents such as antisense oligonucleotides or other nucleobase polymers and medical uses related thereto. In certain embodiments, the phosphate membrane nanodiscs comprise a phospholipid having a thiol group used for conjugation to agents such as oligonucleotides or other nucleobases polymers having a thiol reactive group. In certain embodiment, the phosphate membrane nanodiscs comprise a stabilizing peptide having a thiol group used for conjugation to therapeutic agents such as oligonucleotides or other nucleobase polymers.

In certain embodiments, this disclosure relates to phosphate membrane nanodisc covalently conjugated to a therapeutic agent, wherein the phosphate membrane nanodisc comprises a zwitterionic phospholipid, a nanodisc stabilizing peptide, such as an ApoA1, variant, or fragment thereof, and wherein the therapeutic agent is conjugated to a phospholipid providing a thiol-linked adduct.

In certain embodiments, this disclosure relates to phosphate membrane nanodiscs covalently conjugated to a therapeutic agent, wherein the phosphate membrane nanodisc comprises a zwitterionic phospholipid, a nanodisc stabilizing peptide, such as an ApoA1, variant, or fragment thereof, comprising a C-terminal thiol group, C-terminal cysteine amino acid, a GC sequence, or GGC sequence, wherein the therapeutic agent is conjugated to a phospholipid providing a thiol-linked adduct; and wherein the therapeutic agent is conjugated to the stabilizing peptide providing a thiol-linked adduct to the C-terminal thiol group, C-terminal cysteine amino acid, a GC sequence, or GGC sequence. In certain embodiments, the therapeutic agent is a nucleobase polymer and the thiol-linked adduct is a thiol-maleimide adduct.

In certain embodiments, this disclosure relates to phosphate membrane nanodiscs wherein the nanodisc stabilizing peptide comprises the amino acid sequence of PVLDLFRELLNELLEALKQKLKGGC (SEQ ID NO: 2) and the therapeutic agent is a nucleobase polymer comprising the sequence TGGCAAGCATCCTGTA (SEQ ID NO: 5).

In certain embodiments, a phosphate membrane nanodisc is conjugated to 12, 13, or greater nucleobase polymers on each phosphate membrane nanodisc, wherein the phosphate membrane nanodisc has a diameter of about between 8 and 17 nm, or 11 and 17 nm.

In certain embodiments, the phosphate membrane nanodisc is made by the process of contacting the zwitterionic phospholipid, a phospholipid having a thiol group, and a nanodisc stabilizing peptide, wherein the molar ratio or weight ratio of the zwitterionic phospholipid and the phospholipid having a thiol group is between 95 to 5 and 90 to 10.

In certain embodiments, the phosphate membrane nanodisc is made by the process of contacting the zwitterionic phospholipid, a phospholipid having a thiol group, and a nanodisc stabilizing peptide, at a temperature between 40 and 45 degrees Celsius or between 35 and 55 degrees Celsius.

In certain embodiments, the phosphate membrane nanodisc is made by the process of contacting the zwitterionic phospholipid, a phospholipid having a thiol group, and a nanodisc stabilizing peptide, in an aqueous solution at a pH between 7.5 and 8.5 or between 7.0 and 9.0.

In certain embodiments, the zwitterionic phospholipid is 1,2-dimyristoyl-sn-glycero-3-phosphocholine (DMPC).

In certain embodiments, the phospholipid having a thiol group is 1,2-dipalmitoyl-sn-glycero-3-phosphothioethanol.

In certain embodiments, the molar ratio or weight ratio of the zwitterionic phospholipid to the phospholipid having a thiol group is between 8:1 and 10:1 or between 8:1 and 20:1.

In certain embodiments, the nanodisc stabilizing peptide comprises a C-terminal thiol group optionally conjugated to the peptide by a linking group, cysteine amino acid, a GC sequence, or GGC sequence.

In certain embodiments, the nanodisc stabilizing peptide comprises the amino acid sequence of PVLDLFRELLNELLEALKQKLKGGC (SEQ ID NO: 2).

In certain embodiments, the therapeutic agent is an antisense oligonucleotide. In certain embodiments, the antisense oligonucleotide comprises TGGCAAGCATCCTGTA (SEQ ID NO: 5).

In certain embodiments, the phosphate membrane nanodisc comprises a cationic and/or zwitterionic phospholipid, a phospholipid having a thiol group, a stabilizing peptide, and a therapeutic agent, such as an anti-sense oligonucleotide (ASO) or other nucleobase polymer. In certain embodiments, the cationic an/or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in the ratio of 9:1 or between 8:1 and 10:1. In certain embodiments, the stabilizing peptide sequence is PVLDLFRELLNELLEALKQKLK (SEQ ID NO: 1).

In certain embodiments, the phosphate membrane nanodiscs comprise a cationic and/or zwitterionic phospholipid, a phospholipid having a thiol group, a stabilizing peptide having a thiol group, and a therapeutic agent, such as an anti-sense oligonucleotide (ASO) or other nucleobase polymer. In certain embodiments, the cationic and/or or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in the ratio of 9:1 or between 8:1 and 10:1. In certain embodiments, the stabilizing peptide having a thiol group comprises the amino acid sequence of PVLDLFRELLNELLEALKQKLKGGC (SEQ ID NO: 2), CGGPVLDLFRELLNELLEALKQKLK (SEQ ID NO: 3) or CPVLDLFRELLNELLEALKQKLKC (SEQ ID NO: 4).

In certain embodiments, the phosphate membrane nanodisc comprise a cationic and/or zwitterionic phospholipid, a phospholipid having a thiol group, a stabilizing peptide having a thiol group, and a therapeutic agent, such as an anti-sense oligonucleotide (ASO) or other nucleobase polymer. In certain embodiments, the cationic and/or or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in the ratio of 9:1 or between 8:1 and 10:1. In certain embodiments, the stabilizing peptide having a thiol group comprises the amino acid sequence of PVLDLFRELLNELLEALKQKLKGGC (SEQ ID NO: 2), CGGPVLDLFRELLNELLEALKQKLK (SEQ ID ID NO: 3) CPVLDLFRELLNELLEALKQKLKC (SEQ ID NO: 4) or variants thereof, e.g., those comprising one, two, three, four, or more amino acid substitutions, conserved substitutions, deletions, additions, or combinations thereof. Examples include variants reported in US Patent Publication No. 2018/0250419, hereby incorporated by reference.

In certain embodiments, the therapeutic agent is an anti-sense oligonucleotide (ASO) or other nucleobase polymer which is covalently conjugated to the nanodisc through the phospholipid having a thiol group on the outer surface of the phosphate membrane nanodisc. In certain embodiments, the therapeutic agent or ASO is linked to the phosphate membrane nanodisc through the phospholipid having a thiol group providing a maleimide-thiol adduct. In certain embodiments, the therapeutic agent is an anti-sense oligonucleotide (ASO) or other nucleobase polymer which is covalently conjugated to the phosphate membrane nanodisc through the stabilizing peptide having a thiol group on the outer surface of the nanodiscs. In certain embodiments, the therapeutic agent, ASO, or nucleobase polymer is linked to the phosphate membrane nanodisc through the stabilizing peptide having a thiol group providing a maleimide-thiol adduct.

In certain embodiments, the maleimide-thiol adduct can be conjugated to the therapeutic agent, ASO, or nucleobase polymer using any type of linking group. In certain embodiments, as an alternative to using a maleimide thiol reactive agent, one can use any construct that is reactive with thiol groups (thiol reactive entities) conjugated to the therapeutic agent, ASO, or nucleobase polymer by a linking group. In certain embodiments, thiol reactive entities include haloacetyl, bromoacetyl, or iodoacetyl chemical groups which form thiol ether adducts, and pyridyl disulfides to form disulfide adducts.

In certain embodiments, the anti-sense oligonucleotide or other nucleobase polymer specifically binds to HIF-1-alpha mRNA. In certain embodiments, the anti-sense oligonucleotide or other nucleobase polymer has the nucleotide sequence of TGGCAAGCATCCTGTA (SEQ ID NO: 5). In certain embodiments, the therapeutic agent, antisense nucleotide or other nucleobase polymer comprises 4-(N-maleimidomethyl)cyclohexane-1-carboxamide group or other thiol reactive entity.

### Methods of Use

In certain embodiments, this disclosure relates to methods of treating diseases or conditions comprising administering to a subject in need thereof an effective amount of a phosphate membrane nanodisc as disclosed herein comprising a therapeutic agent or oligonucleotide that can treat the disease or conditions, e.g., a therapeutic agent can specifically bind a disease or condition associated biomolecule.

In certain embodiments, this disclosure relates to methods of treating a disease or condition comprising administering an effective amount of a phosphate membrane nanodisc covalently conjugated to a therapeutic agent as reported herein to a subject in need thereof. In certain embodiments, this disclosure relates to methods of treating cancer comprising administering an effective amount of a phosphate membrane nanodisc covalently conjugated to an anti-sense oligonucleotide or other nucleobase polymer that specifically binds to HIF-1-alpha mRNA as reported herein to a subject in need thereof.

In certain embodiments, this disclosure relates to methods of treating cancer comprising administering to a subject in need thereof an effective amount of a phosphate membrane nanodisc as disclosed herein comprising a nucleobase polymer that specifically binds HIF-1-alpha mRNA. In certain embodiments, the nucleobase polymer comprises TGGCAAGCATCCTGTA (SEQ ID NO: 5). In certain embodiments, the cancer is pancreatic cancer, liver cancer, kidney cancer, lung cancer, non-small cell lung cancer, or small cell lung cancer. In certain embodiments, the phosphate membrane nanodiscs comprise a cationic and/or zwitterionic phospholipid, a phospholipid having a thiol group, a stabilizing peptide having a thiol group, and a therapeutic agent, such as an anti-sense oligonucleotide (ASO) or other nucleobase polymer. In certain embodiments, the cationic and/or or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in the ratio of 9:1 or between 8:1 and 10:1. In certain embodiments, the stabilizing peptide having a thiol group comprises the amino acid sequence of PVLDLFRELLNELLEALKQKLKGGC (SEQ ID NO: 2).

In certain embodiments, this disclosure relates to methods of treating diseases or conditions associated with HIF-1-alpha targeting due to abnormal levels of HIF-1-alpha such as atherosclerosis, psoriasis, diabetic retinopathy, macular degeneration, rheumatoid arthritis, asthma, inflammatory bowel disease, warts, allergic dermatitis, inflammation, and skin inflammation. In certain embodiments, the nucleobase polymer comprises TGGCAAGCATCCTGTA (SEQ ID NO: 5). In certain embodiments, the cancer is pancreatic cancer, liver cancer, kidney cancer, lung cancer, non-small cell lung cancer, or small cell lung cancer. In certain embodiments, the phosphate membrane nanodiscs comprise a cationic and/or zwitterionic phospholipid, a phospholipid having a thiol group, a stabilizing peptide having a thiol group, and a therapeutic agent, such as an anti-sense oligonucleotide (ASO) or other nucleobase polymer. In certain embodiments, the cationic and/or or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in the ratio of 9:1 or between 8:1 and 10:1. In certain embodiments, the stabilizing peptide having a thiol group comprises the amino acid sequence of PVLDLFRELLNELLEALKQKLKGGC (SEQ ID NO: 2).

In certain embodiments, the therapeutic agent is an anti-sense oligonucleotide (ASO) or other nucleobase polymer which is covalently conjugated the phosphate membrane nanodiscs through the phospholipid having a thiol group on the outer surface. In certain embodiments, the therapeutic agent or ASO is linked to the nanodisc through the phospholipid having a thiol group providing a maleimide-thiol adduct. In certain embodiments, the therapeutic agent is an anti-sense oligonucleotide (ASO) or other nucleobase polymer which is covalently conjugated to the phosphate membrane nanodiscs through the stabilizing peptide having a thiol group on the outer surface of the nanodiscs. In certain embodiments, the therapeutic agent, ASO, or nucleobase polymer is linked to the phosphate membrane nanodisc through the stabilizing peptide having a thiol group providing a maleimide-thiol adduct.

In certain embodiments, the therapeutic agent, antisense nucleotide or other nucleobase polymer comprises 4-(N-maleimidomethyl)cyclohexane-1-carboxamide and upon reaction results in a 4-((3-mercapto-2,5-dioxopyrrolidin-1-yl)methyl)cyclohexane-1-carboxamide linking group.

In certain embodiments, the anti-sense oligonucleotide or other nucleobase polymer has the nucleotide sequence of fomivirsen, GCGTTTGCTCTTCTTCTTGCG (SEQ ID NO: 6). In certain embodiments, this disclosure contemplates use of the related phosphate membrane nanodisc for treating cytomegalovirus retinitis (CMV) in immunocompromised patients, including those with AIDS. In certain embodiments, the phosphate membrane nanodiscs comprise a cationic and/or zwitterionic phospholipid, a phospholipid having a thiol group, a stabilizing peptide having a thiol group, and a therapeutic agent, such as an anti-sense oligonucleotide (ASO) or other nucleobase polymer. In certain embodiments, the cationic and/or or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in the ratio of 9:1 or between 8:1 and 10:1. In certain embodiments, the stabilizing peptide having a thiol group comprises the amino acid sequence of PVLDLFRELLNELLEALKQKLKGGC (SEQ ID NO: 2).

In certain embodiments, the anti-sense oligonucleotide or other nucleobase polymer has the nucleotide sequence of pegaptanib, CGGAAUCGUGAAUGCUUAUACAUCCG (SEQ ID NO: 7). In certain embodiments, this disclosure contemplates use of the related phosphate membrane nanodisc for treating neovascular (wet) age-related macular degeneration (AMD). In certain embodiments, the phosphate membrane nanodiscs comprise a cationic and/or zwitterionic phospholipid, a phospholipid having a thiol group, a stabilizing peptide having a thiol group, and a therapeutic agent, such as an anti-sense oligonucleotide (ASO) or other nucleobase polymer. In certain embodiments, the cationic and/or or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in the ratio of 9:1 or between 8:1 and 10:1. In certain embodiments, the stabilizing peptide having a thiol group comprises the amino acid sequence of PVLDLFRELLNELLEALKQKLKGGC (SEQ ID NO: 2).

In certain embodiments, the anti-sense oligonucleotide or other nucleobase polymer has the nucleotide sequence of mipomersen, GCCUCAGTCTGCTTCGCACC (SEQ ID NO: 8). In certain embodiments, this disclosure contemplates use of the related phosphate membrane nanodisc for treating of homozygous familial hypercholesterolemia. In certain embodiments, the phosphate membrane nanodiscs comprise a cationic and/or zwitterionic phospholipid, a phospholipid having a thiol group, a stabilizing peptide having a thiol group, and a therapeutic agent, such as an anti-sense oligonucleotide (ASO) or other nucleobase polymer. In certain embodiments, the cationic and/or or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in the ratio of 9:1 or between 8:1 and 10:1. In certain embodiments, the stabilizing peptide having a thiol group comprises the amino acid sequence of PVLDLFRELLNELLEALKQKLKGGC (SEQ ID NO: 2).

In certain embodiments, the anti-sense oligonucleotide or other nucleobase polymer has the nucleotide sequence of defibrotide aptamers, GGTTGGATTGGTTGG (SEQ ID NO: 9) and/or GGTTGGATCGGTTGG (SEQ ID NO: 10). In certain embodiments, this disclosure contemplates use of the related phosphate membrane nanodisc for treating or preventing the formation of blood clots. In certain embodiments, the phosphate membrane nanodiscs comprise a cationic and/or zwitterionic phospholipid, a phospholipid having a thiol group, a stabilizing peptide having a thiol group, and a therapeutic agent, such as an anti-sense oligonucleotide (ASO) or other nucleobase polymer. In certain embodiments, the cationic and/or or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in the ratio of 9:1 or between 8:1 and 10:1. In certain embodiments, the stabilizing peptide having a thiol group comprises the amino acid sequence of PVLDLFRELLNELLEALKQKLKGGC (SEQ ID NO: 2).

In certain embodiments, the anti-sense oligonucleotide or other nucleobase polymer has the nucleotide sequence of eteplirsen, CTCCAACATCAAGGAAGATGGCATTTCTAG (SEQ ID NO: 11). In certain embodiments, this disclosure contemplates use of the related phosphate membrane nanodisc for treating Duchenne muscular dystrophy (DMD). In certain embodiments, the phosphate membrane nanodiscs comprise a cationic and/or zwitterionic phospholipid, a phospholipid having a thiol group, a stabilizing peptide having a thiol group, and a therapeutic agent, such as an anti-sense oligonucleotide (ASO) or other nucleobase polymer. In certain embodiments, the cationic and/or or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in the ratio of 9:1 or between 8:1 and 10:1. In certain embodiments, the stabilizing peptide having a thiol group comprises the amino acid sequence of PVLDLFRELLNELLEALKQKLKGGC (SEQ ID NO: 2).

In certain embodiments, the anti-sense oligonucleotide or other nucleobase polymer has the nucleotide sequence of nusinersen, TCACTTTCATAATGCTGG (SEQ ID NO: 12). In certain embodiments, this disclosure contemplates use of the related phosphate membrane nanodisc for treating spinal muscular atrophy (SMA). In certain embodiments, the phosphate membrane nanodiscs comprise a cationic and/or zwitterionic phospholipid, a phospholipid having a thiol group, a stabilizing peptide having a thiol group, and a therapeutic agent, such as an anti-sense oligonucleotide (ASO) or other nucleobase polymer. In certain embodiments, the cationic and/or or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in the ratio of 9:1 or between 8:1 and 10:1. In certain embodiments, the stabilizing peptide having a thiol group comprises the amino acid sequence of PVLDLFRELLNELLEALKQKLKGGC (SEQ ID NO: 2).

In certain embodiments, the anti-sense oligonucleotide or other nucleobase polymer has the nucleotide sequence of inotersen TCTTGGTTACATGAAATCCC (SEQ ID NO: 13). In certain embodiments, this disclosure contemplates use of the related phosphate membrane nanodisc for treating polyneuropathy (nerve disease) of hereditary transthyretin-mediated amyloidosis. In certain embodiments, the phosphate membrane nanodiscs comprise a cationic and/or zwitterionic phospholipid, a phospholipid having a thiol group, a stabilizing peptide having a thiol group, and a therapeutic agent, such as an anti-sense oligonucleotide (ASO) or other nucleobase polymer. In certain embodiments, the cationic and/or or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in the ratio of 9:1 or between 8:1 and 10:1. In certain embodiments, the stabilizing peptide having a thiol group comprises the amino acid sequence of PVLDLFRELLNELLEALKQKLKGGC (SEQ ID NO: 2).

In certain embodiments, the anti-sense oligonucleotide or other nucleobase polymer has the nucleotide sequence of golodirsen, GTTGCCTCCGGTTCTGAAGGTGTTC (SEQ ID NO: 14). In certain embodiments, this disclosure contemplates use of the related phosphate membrane nanodisc for treating Duchenne muscular dystrophy (DMD). In certain embodiments, the phosphate membrane nanodiscs comprise a cationic and/or zwitterionic phospholipid, a phospholipid having a thiol group, a stabilizing peptide having a thiol group, and a therapeutic agent, such as an anti-sense oligonucleotide (ASO) or other nucleobase polymer. In certain embodiments, the cationic and/or or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in the ratio of 9:1 or between 8:1 and 10:1. In certain embodiments, the stabilizing peptide having a thiol group comprises the amino acid sequence of PVLDLFRELLNELLEALKQKLKGGC (SEQ ID NO: 2).

In certain embodiments, the anti-sense oligonucleotide or other nucleobase polymer has the nucleotide sequence of viltolarsen, CCTCCGGTTCTGAAGGTGTTC (SEQ ID NO: 15). In certain embodiments, this disclosure contemplates use of the related phosphate membrane nanodisc for treating Duchenne muscular dystrophy (DMD). In certain embodiments, the phosphate membrane nanodiscs comprise a cationic and/or zwitterionic phospholipid, a phospholipid having a thiol group, a stabilizing peptide having a thiol group, and a therapeutic agent, such as an anti-sense oligonucleotide (ASO) or other nucleobase polymer. In certain embodiments, the cationic and/or or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in the ratio of 9:1 or between 8:1 and 10:1. In certain embodiments, the stabilizing peptide having a thiol group comprises the amino acid sequence of PVLDLFRELLNELLEALKQKLKGGC (SEQ ID NO: 2).

In certain embodiments, the anti-sense oligonucleotide or other nucleobase polymer has the nucleotide sequence of casimersen, CAATGCCATCCTGGAGTTCCTG (SEQ ID NO: 16). In certain embodiments, this disclosure contemplates use of the related phosphate membrane nanodisc for treating Duchenne muscular dystrophy (DMD). In certain embodiments, the phosphate membrane nanodiscs comprise a cationic and/or zwitterionic phospholipid, a phospholipid having a thiol group, a stabilizing peptide having a thiol group, and a therapeutic agent, such as an anti-sense oligonucleotide (ASO) or other nucleobase polymer. In certain embodiments, the cationic and/or or zwitterionic phospholipid is 1,2-dimyristoyl-sn-glycero-3-phosphocholine (DMPC). In certain embodiments, the phospholipid having a thiol group is 1,2-dipalmitoyl-sn-glycero-3-phosphothioethanol on an outer surface which provide a thiol reactive phospholipid. In certain embodiments, the 1,2-dimyristoyl-sn-glycero-3-phosphocholine (DMPC) and 1,2-dipalmitoyl-sn-glycero-3-phosphothioethanol are in the ratio of 9:1 or between 8:1 and 10:1. In certain embodiments, the stabilizing peptide having a thiol group comprises the amino acid sequence of PVLDLFRELLNELLEALKQKLKGGC (SEQ ID NO: 2).

### Pharmaceutical Compositions and Kits

In certain embodiments, this disclosure relates to pharmaceutical compositions and kits comprising phosphate membrane nanodisc as reported herein. In certain embodiments, this disclosure relates to the production of a medicament comprising phosphate membrane nanodisc as reported herein for therapeutic uses reported herein.

In certain embodiments, this disclosure relates to pharmaceutical compositions comprising phosphate membrane nanodiscs conjugated to therapeutic agents as reported herein. In certain embodiments, the pharmaceutical composition optionally comprises a pharmaceutical carrier, and that the pharmaceutical composition optionally comprises further therapeutic agents, anticancer agents, anti-inflammatory agents, etc. In certain embodiments, a pharmaceutical composition is in the form of a liquid comprising pH buffering agents and optionally salts and/or saccharide or polysaccharide.

In certain embodiments, this disclosure contemplates an intravenous formulation with pH buffering agents and tonicity in a range representing physiological values (pH 7 to 8) or for bolus administration, e.g., containing normal saline or dextrose optionally containing pH buffering agents. In certain embodiments, the pharmaceutical composition is in the form of a sterilized pH buffered aqueous salt solution or a saline phosphate buffer between a pH of 6 to 8, optionally comprising a saccharide or polysaccharide.

In certain embodiments, this disclosure relates to pharmaceutical compositions comprising phosphate membrane nanodiscs conjugated to therapeutic agents as reported herein and a pharmaceutically acceptable excipient. In certain embodiments, the pharmaceutically acceptable excipient is selected from lactose, sucrose, mannitol, triethyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, ethanol, polyols (propylene glycol, polyethylene glycol, glycerol, and the like), suitable mixtures thereof, vegetable (such as olive oil, sesame oil) and injectable organic esters such as ethyl oleate.

These compositions may also contain preserving, emulsifying, and dispensing agents. Prevention of the action of microorganisms may be controlled by addition of any of various antibacterial and antifungal agents, example, parabens, chlorobutanol, phenol, sorbic acid, and the like. It may also be desirable to include isotonic agents, for example sugars, sodium chloride, and the like. Prolonged absorption of the injectable pharmaceutical form may be brought about by the use of agents delaying absorption, for example, aluminum monostearate and gelatin.

Liquid dosage forms for oral administration include pharmaceutically acceptable emulsions, solutions, suspensions, syrups, capsules, gel capsules, and pills. In addition to the phosphate membrane nanodiscs conjugated to therapeutic agents as reported herein, the liquid dosage forms may contain inert diluents commonly used in the art, such as water or other solvents, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

In certain embodiments, pharmaceutical compositions comprising a phosphate membrane nanodisc disclosed herein can be administered to subjects either orally, parenterally (intravenously, intramuscularly, or subcutaneously), intracisternally, intraperitoneally, intravesically, locally (powders, ointments, or drops), intravaginally, as a buccal or nasal spray, topically to the skin, or rectally.

In certain embodiments, the pharmaceutical compositions are in a form for inhalation. In certain embodiments, the pharmaceutical composition comprises phosphate membrane nanodiscs conjugated to therapeutic agents as reported herein and a propellant. In certain embodiments, an aerosolizing propellant is compressed air, ethanol, nitrogen, carbon dioxide, nitrous oxide, hydrofluoroalkanes (HFAs), or combinations thereof.

In certain embodiments, the disclosure contemplates a pressurized or unpressurized container comprising a phosphate membrane nanodiscs conjugated to therapeutic agents as reported herein. In certain embodiments, the container is a manual pump spray, inhaler, meter-dosed inhaler, dry powder inhaler, nebulizer, vibrating mesh nebulizer, jet nebulizer, or ultrasonic wave nebulizer.

In certain embodiments, this disclosure contemplates kits comprising pharmaceutical compositions comprising phosphate membrane nanodiscs conjugated to therapeutic agents as reported herein and optionally another therapeutic agent/anticancer agent in same or separate pharmaceutical composition or container. The kits may contain a transfer device such a needle, syringe, cannula, capillary tube, pipette, or pipette tip. In certain embodiments, the agents may be contained in a storage container, sealed, or unsealed, such a vial, bottle, ampule, blister pack, or box. In certain embodiments, the kit further comprises written instructions for using the agents for treating and/or preventing cancer or other disease or condition in a subject.

In certain embodiments, this disclosure relates to uses of phosphate membrane nanodiscs disclosed herein in the production of a medicament for treating diseases of conditions disclosed herein.

Dosing is dependent on severity and responsiveness of the disease state to be treated, and the course of treatment lasting from several days to several months, or until a cure is effected or a diminution of the disease state is achieved. Optimal dosing schedules can be calculated from measurements of drug accumulation in the body of the patient. Optimum dosages may vary depending on the relative potency of individual oligonucleotides. Generally, it can be estimated based on amounts found to be effective in in vitro and in vivo animal models. In general, dosage is from 0.01 μg to 1 g per kg of body weight, and may be given once or more daily, weekly, monthly, or yearly, or even once every 2 to 10 years or by continuous infusion for hours up to several months. The repetition rates for dosing can be estimated based on measured residence times and concentrations of the drug in bodily fluids or tissues. Following successful treatment, it may be desirable to have the patient undergo maintenance therapy to prevent the recurrence of the disease state.

### Gene Regulation Using Nanodiscs Modified with HIF-1-alpha Antisense Oligonucleotides

Hypoxia, oxygen deprivation in living tissue, occurs in many forms of cancer. Tumor-induced hypoxia leads to activation of the hypoxia inducible factor (HIF) signaling pathway, which enhances tumor growth and invasion. Thus, HIF inhibitors are promising anticancer agents. See Fallah et al., HIF Inhibitors: Status of Current Clinical Development, Current Oncology Reports (2019)21:6.

Delivery of nucleic acids can be hindered by multiple factors including nuclease susceptibility, endosome trapping, and clearance. Lipid-based systems are advantageous because of their high biocompatibility and low toxicity. However, many lipid nanoparticle systems still have issues regarding stability, rapid clearance, and cargo leakage. Here, the use of a synthetic nanodisc (ND) scaffold functionalized with an anti-HIF-1-alpha antisense oligonucleotide (ASO) was demonstrated to reduce HIF-1-alpha mRNA transcript levels. ND conjugates were prepared by using a mixture of phosphoglycerolipids with phosphocholine and phosphothioethanol headgroups that self-assemble into approximately 13 by 5 nm diameter discoidal structures upon addition of a 22-amino-acid ApoA1mimetic peptide. Optimized reaction conditions yield 15 copies of the anti-HIF-1-alpha ASO DNA covalently conjugated to the thiolated phospholipids using maleimide thiol chemistry. DNA-ND conjugates are active, nuclease resistant. They rapidly internalized into cells to regulate HIF-1-alpha mRNA levels without the use of transfection agents. DNA-ND uptake is partially mediated through Scavenger Receptor B1 and the ND conjugates show enhanced knockdown of HIF-1-alpha compared to that of the soluble ASOs in multiple cell lines. These experiments indicate that covalently functionalized NDs offer an improved platform for oligonucleotide therapeutics.

Nucleic acid therapeutics have evolved into a highly attractive class of drugs that directly target the genetic basis for disease. Antisense oligonucleotides (ASO) typically comprise less than 20mer DNA or RNA nucleotides complementary to/capable of hybridizing to a target mRNA or other nucleotide sequence. ASO drugs have met setbacks in the clinic, in part, because of two main challenges. The first pertains to the short half-life of these molecules due to the activity of endogenous nucleases. The second major challenge is the highly charged backbone of DNA and RNA polymers that limits penetration across the plasma membrane. To reduce nuclease susceptibility, the phosphate backbone is typically modified with a phosphorothioate (PS) or methyl modifications, while the ribose is often modified with 2′ methoxy or fluoro groups, as well as 2′-4′ cross-links. Inadvertently, the PS modification also leads to protein interactions, which results in increased cellular uptake for tissues. Typically, less than 1% of internalized oligonucleotide drugs reach the cytoplasm of the cell, and most are destroyed or trapped within endosomes. Thus, improvements are needed.

Nascent high-density lipoprotein (HDLs) particles are naturally occurring and play a prominent role in delivering cholesterol to the liver through reverse cholesterol transport. In addition to cholesterol transport, HDL may play a role in transporting and delivering various molecular cargo including nucleic acids (miRNAs) through its non-endocytic mechanism of Scavenger Receptor B1 (SRB1) delivery. Nascent HDL particles are composed of ApoA1, phospholipids, and several other minority component proteins. The structure of HDLs can be recapitulated using specific phospholipids and short ApoA1-mimetic peptides, synthetic nanodiscs (NDs). SRB1 is commonly expressed in many cellular subtypes, hence widening the realm of possibilities for targeted delivery.

Therapeutic oligonucleotides, such as siRNA, DNA, PNA, and miRNA, may be anchored onto NDs using noncovalent linking strategies through cholesterol tagging or electrostatic attraction using NDs assembled with positively charged phospholipids or polylysine. One of the challenges in using ND for oligonucleotide delivery pertains to the labile nature of these interactions, which leads to instability of the conjugates. For example, the observable k_{off} between cholesterol-labeled oligonucleotides and phospholipid membranes offers short half-lives, t_{1/2} of about 1-10 min. Another problem with the noncovalent assembly of ND-nucleic acid conjugates is the low density of oligonucleotides. It is thus desirable to generate covalently linked ND-nucleic acid structures with greater densities of nucleic acids to boost their activity.

A range of bioconjugation methods were tested. It was identified that the maleimide-thiol Michael addition chemistry was the most favorable. By titrating different thiol phospholipid concentrations and various reaction conditions (temperature and pH), one is able to boost the density of oligonucleotides to 15 copies/ND while maintaining the NDs ultrasmall size and monodispersity. Importantly, ND conjugation afforded enhanced nuclease resistance.

The uptake and efficacy of NDs conjugated to a clinically relevant ASO that targets hypoxia inducible factor 1 alpha (HIF-1-alpha) was studied. Experiments were performed to determine whether ASO-NDs are taken up by a variety of cell types and internalization is SRB1-dependent. ASO-NDs were found to knock down HIF-1-alpha in a time- and concentration-dependent manner. On an ASO-basis, the maleimide adduct ND-conjugation approach affords about 3-fold improvement in knockdown of HIF-1-alpha in HeLa cells (75 nM of the ASO, 24 h) when compared to the ASO itself, which represents a marked enhancement in drug efficacy.

### Incorporating and Assembling Thiol-Functionalized NDs

NDs were prepared from small unilamellar vesicles (SUVs) comprised of 1,2-dimyristoyl-sn-glycero-3-phosphocholine (DMPC) and 1,2-dipalmitoyl-sn-glycero-3-phosphothioethanol (referred to as “thiol lipid”). To self-assemble the NDs from the SUVs (1 mL of lipid at 2.5 mg/mL), 751 nM of ApoA1-mimetic peptide, PVLDLFRELLNELLEALKQKLK (SEQ ID NO: 1) was incubated with the SUVs, and the samples were subjected to thermal cycling. In preliminary experiments, strain-promoted Cu-free click reactions were tested to couple nucleic acids to NDs. However, these methods generated low DNA conjugation yields. Coupling between maleimide-activated DNA to a thiolated ND produced the most promising initial yields. Experiments were performed to determine the maximum density of the thiolated lipids that could be incorporated into the ND. A range of molar percentages (5-20%) of thiol lipids were tested. The ND structure was assessed using transmission electron microscopy (TEM). Five (5) mol % and 10 mol % thiol-NDs displayed a monodisperse morphology, whereas the 20 mol % thiol-NDs showed aggregation and broadening of ND size. It is contemplated that the thiolated lipids can lead to the formation of disulfide bonds, prompting aggregation.

The thiolated lipids have a longer lipid tail which may contribute to the observed aggregation. Quantification by TEMs indicated that ND diameters slightly increased with increasing thiol lipid content, though the values are not significant. For example, the 0% thiol ND had a diameter of 11.9±2.8 nm, and this increased to 12.1 nm±2.3 nm for 5 mol % thiol ND, and 12.5 nm±1.8 nm for the 10 mol % thiol NDs. The ND thickness seemed independent of thiol concentration and was approximately 4.4, 4.5, and 4.7 nm for the 0%, 5%, and 10% thiol lipid ND particles respectively. The ND thickness and diameter were consistent with the reported dimensions for DMPC bilayers and other NDs generated confirming that the ND structures formed appropriately and likely adopted the structural belt-like conformation. The top and side views of NDs were distinctly visualized confirming the disc-like configuration typical of nascent native HDL and NDs. ND “coin-like” stacks were also observed. This phenomenon is ascribed as the “rouleaux effect”, a common artifact of the negative staining process in TEM due to the interaction of the negatively charged particles in the stain with the choline headgroups during the drying process.

NDs were self-assemble from small unilamellar vesicles (SUVs) composed of 90% DMPC and 10% thiolated phospholipids after adding a 22-amino-acid ApoA1mimetic peptide. DNA is covalently conjugated to the surface of the ND using thiol-maleimide coupling. TEM was used to determine the maximum thiolated phospholipid content that can be tolerated and thus help achieve NDs with up to 15 DNA copies per ND. Deoxyribozyme-ND conjugates are functional and are partially protected from DNase activity when compared to soluble oligonucleotides. ASO-ND conjugates are internalized by cells and show time and concentration dependent uptake. Dual-tagged ASO-ND conjugates display reduced colocalization as a function of time in cells and hence confirm separation of the ASO and phospholipid components over time. ASO-ND conjugates selective for HIF-1-alpha showed greater activity than that of the soluble ASO drug, without the use of transfection agents and across a panel of three cell lines. ND can be engineered to deliver multiple cargos including miRNAs and siRNA as well as lipophilic molecules and peptides, and hence this platform has broad applications as a therapeutic.

### Synthesis and Characterization of Thiol ND

DMPC and the thiol lipid stocks were combined with chloroform (90:10, 80:20, and 95:5 molar ratios) and placed on a rotary evaporator to dry for 1 h. Cy5-PE was doped in at a molar ratio of 0.15% when necessary for certain experiments. After 1 h, the lipid mixture was placed under a steady stream of nitrogen for 10 min prior to hydrating the lipid film with phosphate buffered saline (PBS, pH 7.4). The mixture was sonicated for 10 min before subjecting it to three freeze-thaw cycles. SUVs were subsequently prepared by passing the mixture 10 times through a 10 mL LIPEX Thermobarrel™ extruder using an 80 nm polycarbonate filter. The ApoA1mimetic peptide (2 mg) was dissolved in water and added to the SUVs prior to vortexing the mixture for 30 s. The mixture was subjected to three warm-cool cycles alternating between 55° C. and 4° C. for 15 min each. The thiol-NDs were stored at 4° C. for up to 3 weeks.

### ND Conjugation to DNA

To facilitate a maleimide-thiol linkage onto the surface of ND, an amine-terminal DNA was modified with a maleimide group bearing a heterobifunctional linker, succinimidyl 4-(N-maleimidomethyl) cyclohexane-1-carboxylate (SMCC). DNA-ND conjugates were prepared using a deoxyribozyme (DNAzyme) which has catalytic activity that is highly sensitive to the local environment. The maleimide activated DNAzyme (DNA) was then coupled to the surface of NDs which were first treated with tris(2-carboxyethyl)phosphine (TCEP) to reduce the thiols. The DNA density on NDs was measured and compared densities for NDs displaying 5 and 10 mol % thiol lipids under standard reaction conditions (RT, pH 7.4, 2 h). NDs composed of 10 mol % thiol lipids displayed a greater DNA density compared to the 5 mol %. The coupling conditions were at alternate temperatures (25 and 45° C.) and pH (7.4 and 8.5). Elevated temperatures in combination with a more basic pH resulted in an average DNA density (13±2 DNA strands/ND). Note that this coupling strategy and conditions resulted in DNA densities that significantly exceed that of cholesterol tagged siRNAs. DNA modification and exposure to higher temperature and pH conditions did not alter the structure of the ND. The structure-dependent properties of DNA-ND remain intact and still resemble that of discoidal pre-β HDL.

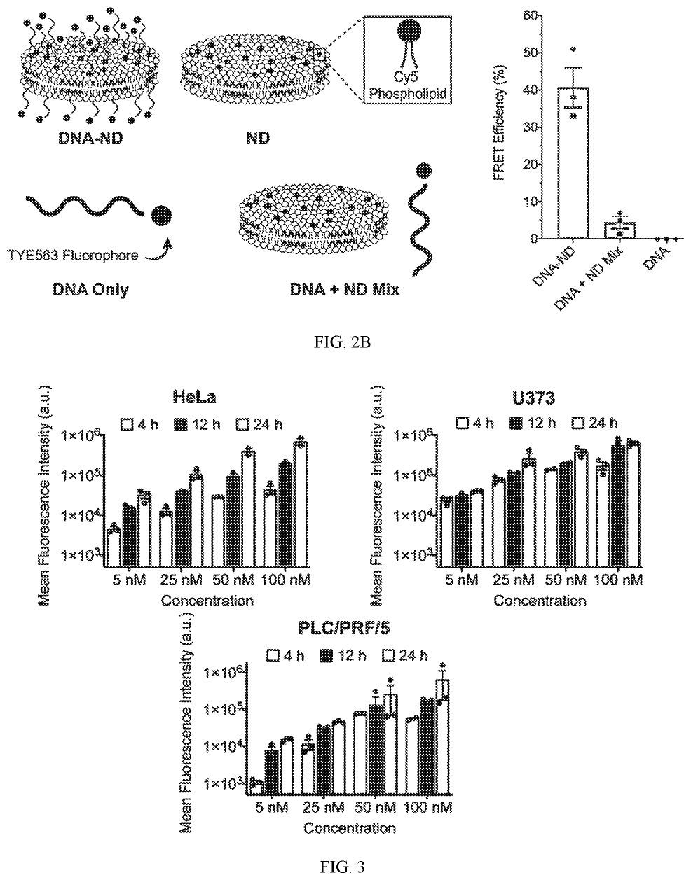

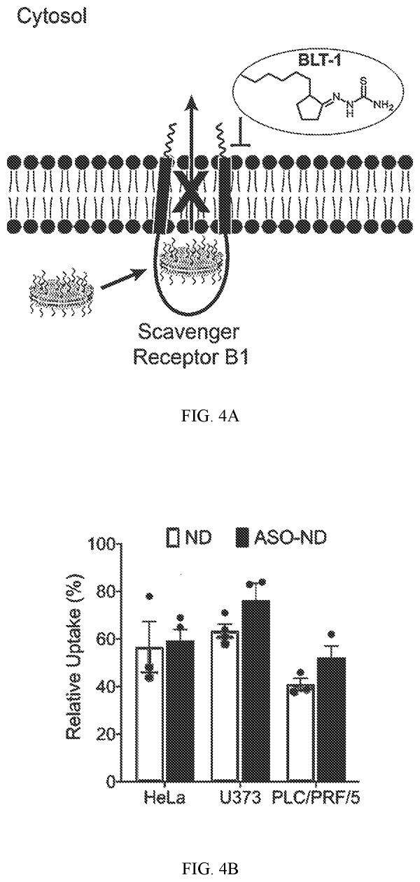

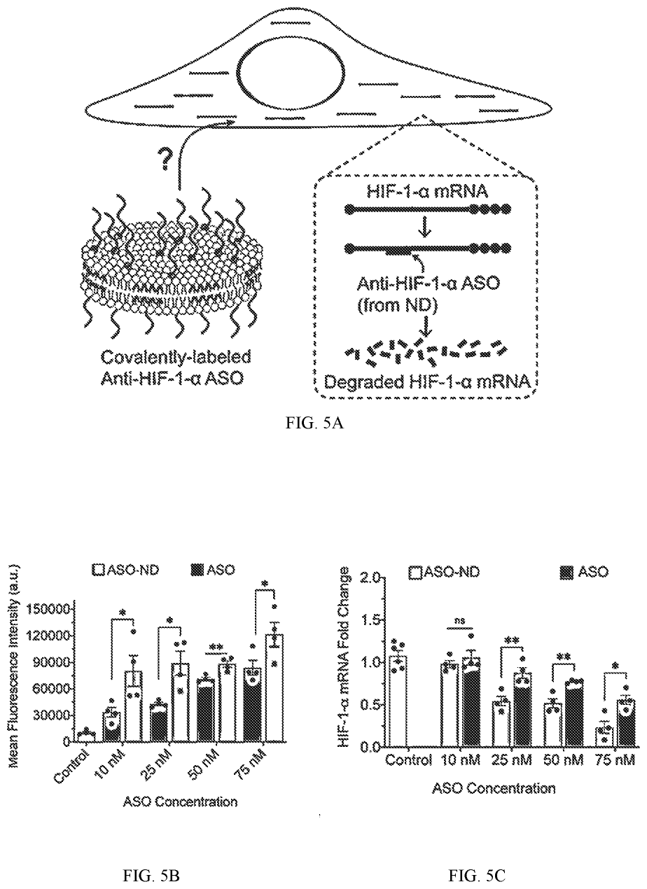

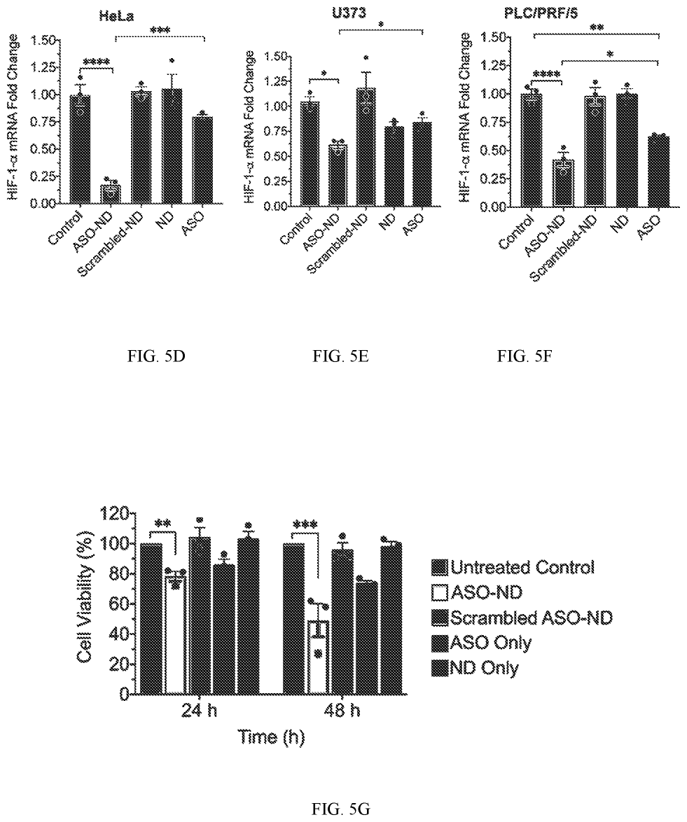

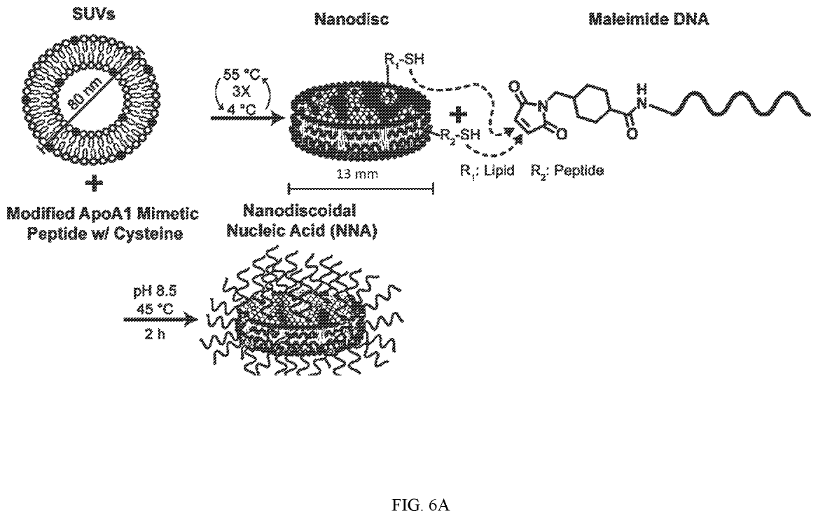

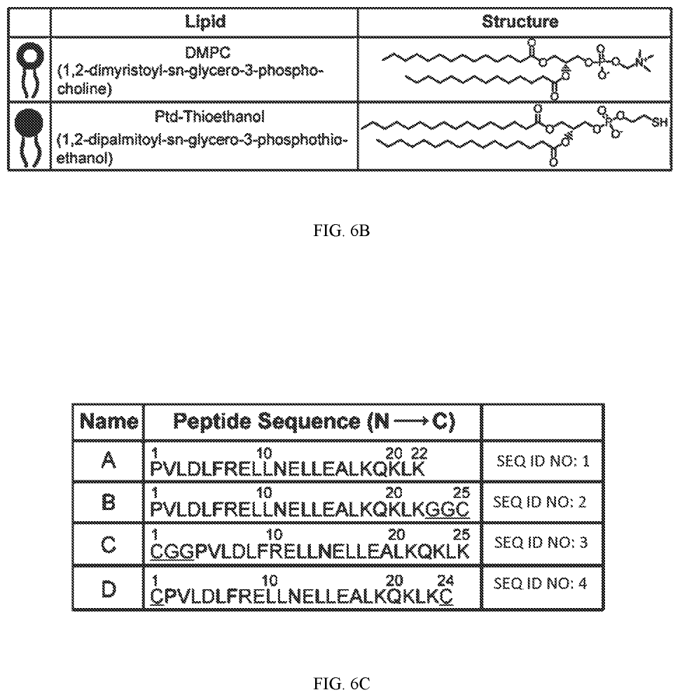

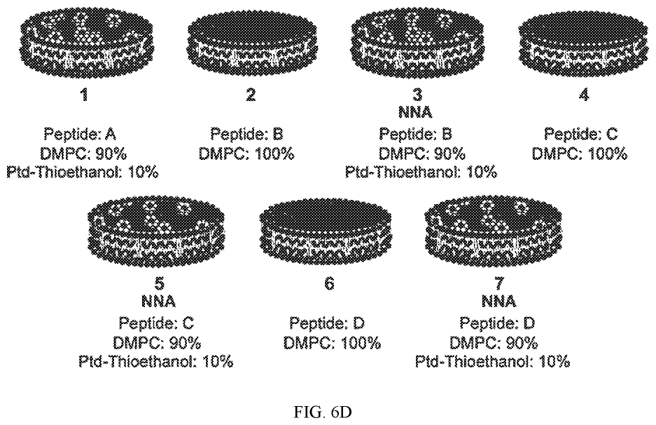

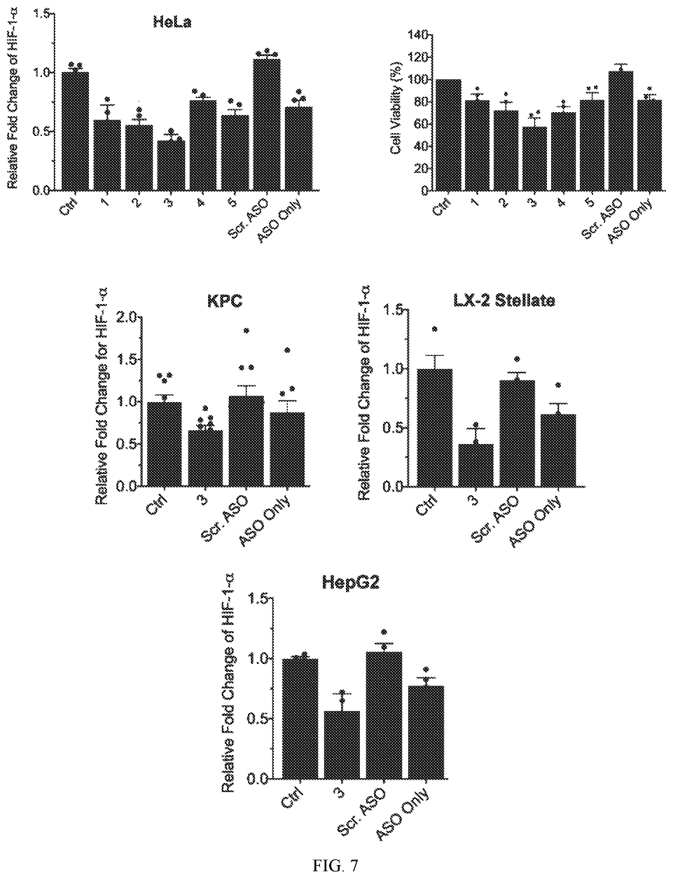

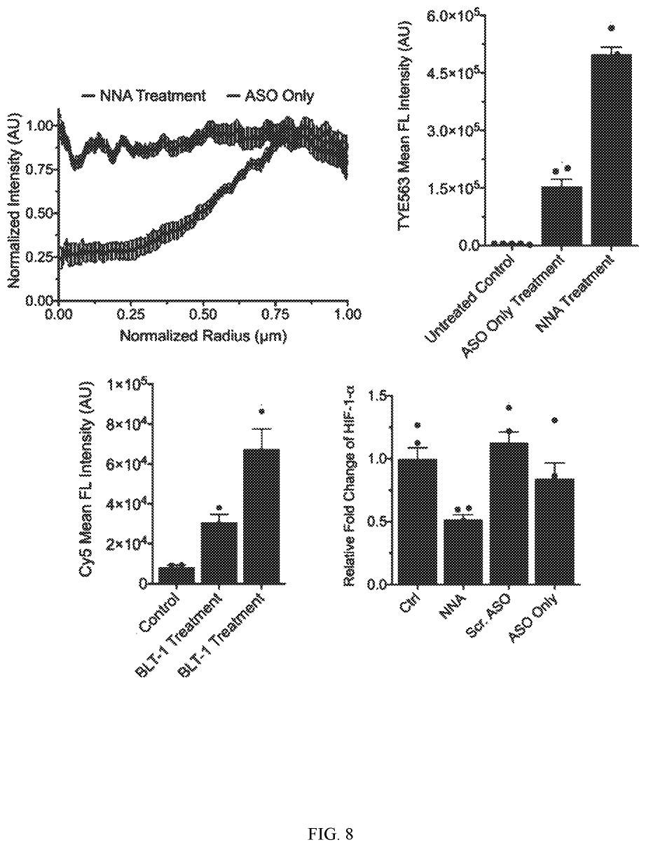

Experiments were performed to determine the DNA conjugation to the ND. DLS indicated a shift in the hydrodynamic radius after coupling with DNA, and the average size of NDs increased from 13 nm±4 nm to 15 nm±7 nm (n=3 independent replicates). Zeta-Potential measurements showed a drastic shift from −10.9 mV to −35.2 mV (FIG. 2b) after DNA conjugation, indicating the presence of negatively charged nucleic acid on the surface of the ND. Agarose gel electrophoresis was used to confirm covalent conjugation of the DNA in samples that had Cy5-tagged lipids. The ND-conjugated DNA showed a marked retardation in migration compared to soluble DNA. In contrast, the ND band migrated more rapidly as a result of DNA conjugation, which is consistent with an increase in charge density as a result of DNA conjugation. Notably, changes in the bands were not observed when DNA was mixed with the ND, indicating weak, if any, electrostatic interactions. FRET measurements further confirmed direct DNA conjugation to the ND. The short anti-HIF-1-alpha ASO was tagged with a TYE563 donor fluorophore while the ND incorporated a Cy5 acceptor fluorophore. Donor emission spectra showed that DNA conjugation led to a significant reduction in donor emission intensity when compared to donor-only sample or samples that mixed the DNA with the ND. The calculated FRET efficiency was 40% for the DNA-ND conjugate and 5% for the mixture of the DNA and ND. The relatively moderate FRET efficiency is because the acceptor is not directly attached to the TYE labeled DNA. Rather, the donor (TYE-DNA) and the acceptor (Cy5 phospholipids) are localized to the same ND, and thus the FRET efficiency reflects the statistically averaged donor-acceptor distance. Collectively, these results establish that the DNA is chemically linked to the ND.

### DNA Bound to the Surface of the ND is Functional and Nuclease-Resistant

A DNAzyme sequence was used in experiments. The activity of the DNAzyme containing the catalytic loop derived from the 10-23 DNAzyme was measured against a fluorogenic substrate. To achieve multiple turnover kinetics, the kinetic measurements employed a 10-fold excess of the substrate compared to the DNAzyme-ND (or soluble DNAzyme). The nucleic acid substrate was dual-labeled with a FAM fluorophore at the 5′ terminus and an Iowa Black quencher at the 3′ terminus. The FAM fluorescence intensity (FL) was monitored over a 4 h time period and fits of these plots provided the k_{obs} rate constants. DNAzyme-ND conjugates displayed about 34% loss in activity compared to the soluble DNAzyme. The ND afforded nuclease resistance, as DNAzyme-ND conjugates retained 64% of their activity after treatment with DNase I for 2 h, while the soluble DNAzyme only preserved approximately 27% of the activity after the same nuclease treatment. Thus, the ND scaffold is a suitable base for delivering therapeutic nucleic acids.

### ASO-ND Conjugates are Internalized in a Dose- and Time-Dependent Manner

To quantify the uptake of ASO-ND conjugates, model cell lines were evaluated. HeLa cells were incubated with anti-HIF-1-alpha ASO, TGGCAAGCATCCTGTA (SEQ ID NO: 5) [5′ end TGG locked nucleic acid (LNA) modifications and 3′ end GTA locked nucleic acid (LNA) modifications and phosphorothioate (PS) backbone modifications] and phospholipid dual-tagged fluorescent ASO-ND conjugates for 3, 12, and 24 h. Then cells were washed and imaged by confocal microscopy. Accumulation of ASO and phospholipid scaffolds were observed inside the cytoplasm of the cells but were excluded from the nucleus. ASO-ND conjugates were primarily localized to the cell edge at 3 h, indicating the cargo was associated with membrane or possibly in endosomes. The Pearson's coefficient for colocalization between the phospholipid and ASO was significantly higher than that measured for control samples containing a mixture of NDs and ASO at all time points. This indicates that a significant subset of ASO-ND conjugates remained intact upon cell uptake. There is a decrease in colocalization at 24 h, indicating disassembly of the ASO-ND conjugates at later time points. the presence of both ASO and ND puncta was observe indicating that there may be multiple populations of ASOs. Possibly a subset of ASOs is entrapped within endosomes, which would appear as puncta. Another population that appears as puncta is possibly in the form of assembled phospholipid-ASO structures that have been internalized using the primary SRB1 uptake pathway for the NDs. Additionally, because PS-modified ASOs are used, it is possible that the DNA was trafficked inside the cell using multiple productive and nonproductive entry pathways that would appear as puncta. Since the DNA is lipidated, puncta may be associated with membranes such as the ER, nuclear membrane, mitochondrial membrane, plasma membrane, and other vesicle-like structures.

Dose-dependent and time-dependent uptake of ASO-ND conjugates into HeLa cells, U373 cells, and PLC/PRF/5 cells were quantified by incubating with fluorescently tagged ND and ASO-ND at varying concentrations (0-100 nM) for different lengths of time (4 h-24 h) prior to analyzing cells by flow cytometry. Both the scaffold and the ASO-ND were taken up by the different cells in a similar dose- and time-dependent manner. At the ASO-ND concentrations tested, saturation of uptake was not observed suggesting that on is able to dose these conjugates at yet greater concentrations to further boost mRNA inhibition.

### Uptake of Thiol NDs and ASO-NDs into Cells is Partially Mediated by Scavenger Receptor B1 (SRB1)

To elucidate the role of SRB1 in uptake, RTqPCR was performed on HeLa, U373, and PLC/PRF/5 cells to confirm the expression of SRB1. SRB1 levels were quantified relative to wild-type Huh7 cells, a known expressor for SRB1, as a positive control. The cell line panel was incubated with an inhibitor for SRB1, blocker of lipid transport-1 (BLT-1) before treatment with ND or ASO-ND for 2 h. Fluorescence intensity was measured using flow cytometry. Fluorescence intensity of the inhibited cells were compared against cells that were treated with ASO-ND or ND but no inhibitor. BLT-1-treated cells displayed reduced uptake compared to the cells treated with ND only, without BLT-1. Notably, ASO-ND displayed more uptake compared to ND only after blocking the SRB1 receptor. This is possibly due to the PS modifications, which mediate internalization through endocytosis by adsorption onto various cellular surface proteins, including SRB and LDL-receptor entry pathways. Hence, the presence of PS modifications may further facilitate the trafficking of ASO-ND inside the cell, especially when conjugated to a delivery vehicle. The uptake of ND and ASO-ND into PLC/PRF/5 liver cells was lower following BLT-1 treatment compared to the uptake in HeLa and U373 cells. Hepatocytes are prominent expressors of SRB1 due to the inherent role of HDL docking and offloading of cholesterol for processing and clearance. Thus, these cells are sensitive to SRB1 blocking. Although it is not intended that embodiments of this disclosure be limited by any particular mechanism, it believed that the trafficking of thiol-NDs into cells is partially mediated by SRB1, a pathway that circumvents endosomal entrapment by selectively taking up and delivering the ND content inside to the cytoplasm of the cells.

### Anti-HIF-1-Alpha ASO-ND Conjugates are Active In Vitro

The transcription factor HIF-1-alpha is sensitive to hypoxia and aids in regulation of responses such as vascularization and angiogenesis that can ultimately tune oxygenation in tissues. HIF-1-alpha also drives survival and adaptation to hypoxic or inflammatory conditions such as that found in solid tumors and in wound healing. Accordingly, there is significant interest in developing drugs that can downregulate the expression of HIF-1-alpha. HIF-1-alpha inhibitors PX-478 and bortezomib are anticancer agents. These inhibitors lack cell or tissue specificity and carry significant off-target effects. Nucleic acid-based drugs that target HIF-1-alpha at the transcript level may show improvements. EZN-2968 is a potent HIF-1-alpha gapmer ASO, designed to bind HIF-1-alpha and induce RNase H cleavage. Use in patients with solid tumors indicates significant reduction of HIF-1-alpha levels. Experiments where performed to evaluate the potency of anti-HIF-1-alpha ASOs upon conjugation to the ND phospholipids and to test whether function is maintained or potentially enhanced compared to the unmodified nucleic acid drug.

HeLa cells were treated for 24 h using different concentrations of ASO-ND and ASO. No transfection agent was used in these experiments and the ASOs were spiked into the media at concentrations that ranged from 10 to 75 nM. A TYE-tagged ASO was used. Flow cytometry was used to quantify the relative uptake levels. Dose-dependent internalization was observed for both ASO and ASO-ND groups. ND-conjugation shows a significant increase in uptake compared to that of the unmodified ASO group. To test ASO function, identical conditions to those used for uptake measurements were applied. HIF-1-alpha levels were measured using RT-qPCR. Dose-dependent knockdown of HIF-1-alpha was observed for both the ASO and ASO-ND groups. Conjugation to the ND resulted in increased cellular internalization and increased reduction in HIF-1-alpha compared to the bare ASO. Knockdown levels were normalized by the uptake levels to estimate the effective activity of ASO when delivered in the unmodified and ND forms. The ND conjugation increased the potency of ASOs on a per molecule basis. ND conjugation may lead to more productive pathways of uptake, such as HSPG and SRB1 mediated internalization, that allow the ASO to access the cytoplasm and the target mRNA.

Three cell lines (HeLa, U373, and PLC/PRF/5) were treated with ASO, ASO-ND, scrambled-ND, and ND for 24 h. HIF-1-alpha levels were measured using RT-qPCR. These three cancer cell lines were selected because of their high intrinsic expression of HIF-1-alpha and their diverse source tissues. The ASO concentration was maintained in all groups to 75 nM. The regulation of HIF-1-alpha was specific to the ASO. No knockdown was observed with the scrambled sequence. The ASO-ND group showed greater levels of HIF-1-alpha knockdown when compared to the ASO only group across the three cell lines tested. SRB1 expression was poorly correlated with ASO-ND activity, as SRB1 expression followed this trend PLC/PRF/5>U373>HeLa while ASO-ND knockdown efficiency followed this trend HeLa>PLC/PRF/5>U373. Given that the uptake of NDs and knockdown efficiency of the unconjugated ASOs was similar in all three cell lines (approximately 25%); this suggests other mechanisms of enhanced uptake specifically for the ASO-ND conjugates in HeLa cells. Delivery of ASOs using NDs is found to be highly advantageous for modulating gene expression levels in vitro.

HIF-1-alpha is important for cancer cell survival and proliferation, and its knockdown can reduce cell survival. Therefore, the functional activity of the ASO was further confirmed by measuring cell viability. In this experiment, HeLa cells were treated for 24 and 48 h, and then cell viability was measured using the MTT assay. Five (5) groups were included: ASO-ND, Scrambled ASO-ND, ND only, and ASO only. Across three independent experiments and at the 24 and 48 h time points, the most significant decrease in cell viability was observed for cells treated with the ASO-ND compared to untreated cells or to cells treated with scrambled ASO-ND. The soluble ASO also showed a decrease in cell viability at 24 and 48 h. These results are consistent with the HIF-1-alpha knockdown levels which showed that the ASO-ND was more active compared to the soluble ASO.

### Nanodiscoidal Nucleic Acids (NNA)—NDs that Present Nucleic Acids on all Faces of the Structure—Both the Phospholipid Headgroups as Well as the Peptide Perimeter Using the ND Scaffolds

Given that a large percentage of the ND is comprised of the peptide scaffold, one could increase DNA loading by directly linking the nucleic acid to the peptide spanning the perimeter of the ND, i.e., in addition to the lipid components. This may be accomplished by the introduction of a reactive group to the amphipathic peptide which may disrupt its propensity to form ND as subtle and, in some cases, single amino acid modifications have been shown to abolish its activity. Therefore, library of cysteine modified ApoA1-mimetic peptides were screened focusing on a 22 amino acid (22A) target. reactive thiol residues were used because this is a native amino acid which is likely less immunogenic and given that we can introduce thiol-containing phospholipids into the ND, having thiolated peptides suggests the potential for running an efficient one-pot reaction to couple oligonucleotides to ND using Michael addition chemistry. One can create a nanodiscoidal nucleic acid (NNA) with DNA loaded on the perimeter as well as the top and bottom lipid faces of the structure.

Cysteines (Cys) were inserted on the N—, C—, or both termini of the peptide and identified peptides that efficiently formed homogenous populations of NNAs. The optimal NNAs employed C-terminal GGC modified residues and presented an average of 30 copies of DNA per NNA. The dense NNA structure retained its ultrasmall size (˜12 nm) and discoidal morphology and demonstrated significant nuclease and serum stability. An antisense oligonucleotide (ASO), EZN2968, that targets hypoxia inducible factor 1-alpha (HIF-1-alpha) mRNA, was conjugated to the ND. In vitro uptake along with reduction in HIF-1-alpha transcript levels, and a marked decrease in cell viability of cancer cells that are HIF-1-dependent dependent was observed. HIF-1-alpha has a role in promoting survival of cancerous and tumorous tissue. The efficacy of anti-HIF-1-alpha NNAs was further validated using 3D spheroid models that showed enhanced uptake that was SRB1-mediated with an approximately 2-fold enhancement in transcript knockdown compared to identical concentrations of naked ASO. Delivery and activity of anti-HIF-1-alpha NNA conjugates in vivo and specifically in liver and kidney tissues was confirmed using a murine model. Activity at low dosing (0.7 mg/kg) was observed to provide a 5-fold enhancement compared to conventional ASOs.

### Screening of Cysteine-Modified ApoA1 Mimetic Peptides

ND scaffolds were assembled by preparing small unilamellar vesicles (SUVs) through extrusion. SUVs were comprised of phospholipids: 1,2-dimyristoylsn-glycero-3-phosphocholine (DMPC) and/or 1,2-dimyristoyl-sn-glycero-3-phosphothioethanol (Ptd-Thioethanol) in a 90:10 (% molar) ratio. Cy5 headgroup tagged phospholipid was added at a 0.15%-1.0% molar ratio as needed to visualize NDs using fluorescence. An ApoA1mimetic sequence, 22 A, was clinically evaluated in a phase I safety analysis and demonstrated to be tolerable. The NNA scaffolds were created by inserting one or two Cys residues at the N- and/or C-terminus of the 22A peptide to generate NDs with peptides B, C, and D. To conjugate nucleic acids to the top and bottom faces of the ND, 10% thiol modified phospholipids were doped into the lipid layers enabling nucleic acid conjugation to the phospholipids. The peptides containing one Cys insertion at the N- or C-terminus (peptides B and C) also included a double glycine spacer to minimize disruption to the native amphipathic structure of the peptide. The double-Cys insertion (peptide D) into 22A peptide did not contain a double glycine spacer. The peptides were capped with an acetyl group at the N terminus and an amide group at the C-terminus for each of these peptides. Following the ND formation, maleimide-modified DNA was coupled to the scaffold to create DNA-ND and NNA conjugates. Structures that present nucleic acids solely on phospholipids or solely on peptides are referred to as DNA-ND conjugates.

The DNA-ND and NNA conjugates were visualized before and after DNA coupling using transmission electron microscopy (TEM). The imaging revealed a monodisperse, homogenous morphology with “coin-like” stack formations, attributed to the rouleaux effect from the negative staining process, for NDs before and after conjugation to DNA. Furthermore, stacking behavior could be driven by the dehydration of the ND during the sample preparation process and can promote a stacking orientation. In contrast, NDs assembled using peptide D showed heterogenous morphology prior to and after DNA conjugation. The only exception was ND 6 that showed some stack formation prior to DNA conjugation but these were more disorganized than other NDs tested (1-5). The weaker propensity to form intact ND for 6 and 7 is potentially due to the N- and C-Cys modified termini which increase the probability of forming disulfide bridges and aggregation. For NDs 1-5, there was a small (nonsignificant) increase in diameter as measured by TEM, which showed that ND size changing from about 11 between 12 nm before DNA conjugation to about 13 between 15 nm after coupling. DLS measurements showed a much more substantial increase in particle size as the hydrodynamic radius shifted from about 10 between 13 nm before DNA addition to about 14 between 22 nm after DNA addition. Therefore, the TEM and DLS confirm the ultrasmall size of the native scaffold following DNA coupling.

Interestingly, the intensity-normalized DLS data shows an increase in the polydispersity of NDs and specifically the appearance of larger-diameter particles, which was most pronounced for ND 5. This is due to the formation of a small population of liposomal aggregates that likely form due to formation of disulfide bridges between ND during the DNA coupling reaction as well as destabilization of the ND following DNA-coupling. These aggregates were more distinct for NNA (ND 5) rather than the peptide-DNA and phospholipid-DNA conjugate ND suggesting: 1) that high density DNA on the ND can lead to slight destabilization of the ND and 2) that introducing the Cys to the N-terminus of 22A was slightly more destabilizing. Helical wheel projections indicated that the peptide C places the N-terminal Cys, which is considered polar, within the hydrophobic face of the peptide, and may explain the decreased stability of ND 4 and 5 compared to that of ND 2 and 3. NNAs with Cys-modified 22A peptides and thiol containing phospholipids were generated that are monodisperse maintaining an approximately 5 nm by about 13 nm disc-like structure based on TEM.

DNA density was measured on NDs 1-5 using the OliGreen™ assay. NNAs (NDs 3 and 5) had the greatest DNA density per disc. ND 1 only presented thiols on the phospholipid and led to 11 plus/minus 5 DNA strands per ND. ND 2, which displayed Cys at the peptide C-terminus afforded 16 plus/minus 1 DNA strands per ND. ND 3 displayed 25 plus/minus 7 DNA strands per ND which suggests that DNA coupling can efficiently proceed on both the phospholipid and peptide with minimal steric clash. ND 4 displayed a lower density compared to the ND 2. ND 5 showed the largest density of 35 plus/minus 14 DNA strands per ND. The average density of these NNAs was the greatest. ND 3 is attractive as a therapeutic candidate given its enhanced monodispersity and consistent DNA density.

To validate that the DNA is covalently linked to the ND, a series of characterization experiments were further performed on representative DNA-ND and NNA samples using FRET. TYE563 fluorophore tagged ASOs that target HIF-1-alpha were used as the donor. The ND was tagged with an acceptor dye (1% Cy5 headgroup modified phospholipid). The TYE563 donor was excited at 525 nm. The collected the emission spectra was used to quantify sensitized emission from Cy5. Qualitatively, Cy5 emission at 670 nm compared to direct donor emission (560 nm) was greatest for ND 1, 2, and 3 compared to controls where the DNA was not covalently linked to ND or when the ND lacked the Cy5 acceptor or when the donor was absent. FRET efficiency was quantified by using the ratio of donor emission normalized to the donor emission in the absence of the acceptor. Using this analysis, ND 1, 2, and 3 showed greater quantitative FRET (40-70%) compared to that of controls where the ND and DNA were present in the solution but not covalently linked (2%). Interestingly, ND with DNA linked to the peptide showed lower FRET efficiency compared to ND with DNA linkage to the phospholipid.

To further validate the covalent attachment of the DNA to the ND, gel electrophoresis of NDs that were labeled with 0.15% Cy5 phospholipid and coupled to DNA were ran. SYBR Gold emission from the DNA was pseudo colored yellow while the Cy5-phospholipid emission was pseudo colored red. NDs 1-5 that were conjugated to DNA showed different mobility compared to an ND scaffold only, DNA mixed with ND (but unconjugated), and soluble DNA only. Upon DNA conjugation, the ND and associated phospholipid migrated more rapidly through the gel compared to the NDs lacking DNA. Additionally, the DNA mobility was slowed upon ND conjugation. Taken together, the gel electrophoresis results along with the FRET analysis confirm covalent coupling of the DNA to the ND both through peptide as well as phospholipid coupling.

Increasing the density of DNA may lead to enhanced DNase resistance. Experiments were performed to determine whether NNAs demonstrate this phenomenon which would be beneficial for boosting nucleic acid drug efficacy. The stability of the NNA structure (ND 3) was compared to soluble DNA and representative DNA-ND samples (NDs 1 and 2). Deoxyribozymes (DNAzymes) were used because their catalytic activity is easily measurable, their catalytic function is fully recovered after heat inactivation and DNAzyme activity is highly sensitive to cleavage; hydrolysis of a single nucleotide from a DNAzyme leads to detectable changes in enzyme activity. The NNA and DNA-ND samples were exposed to 1 U of DNase I for 2 h prior to inactivating the DNase I and assessing functional multi-turnover kinetics of the DNAzyme using a dual-labeled mock RNA substrate. The NNA structure offered greater nuclease resistance compared to the DNA-ND samples (83% activity retained vs 66% respectively). Notably, the nucleic acid used in this sequence was unmodified nucleobases. These data suggest that the ND scaffold provides enhanced protection against nucleases, further improving its potential for delivering therapeutic nucleic acids.

### NNAs are Internalized into Cells Via Scavenger Receptor B1

EZN2968 HIF-1-alph targeting ASO was tagged with a 5′ TYE563 while the ND was labeled with 1% Cy5 to aid in quantifying cell uptake using confocal microscopy. HeLa cells were incubated with 100 nM (with respect to DNA concentration) of representative ASO-ND (1-2) and NNA (3) samples and then imaged at 3 h and 24 h timepoints. A time-dependent increase in accumulation of the NNAs and ASO-NDs were observed by the increased signal for ND 1, 2, and 3 in confocal imaging of single cells. Also, the lipid-Cy5 and DNA-TYE563 signals generally became more dispersed, less colocalized and less punctate at 24 h, as shown in the images and representative line-scans. These observations suggest escape of these conjugates into the cytoplasm at later time points. In contrast, at the early 3 h timepoint, the NNAs and ASO-NDs displayed lower total signal and more punctate clusters where the TYE563 signal was colocalize to the Cy5 signal. Moreover, the TYE563 and Cy5 signals tended to localize towards the cell edge at early time points suggesting that a fraction of the ND is docked to the membrane or internalized and trapped inside endosomes that are near the membrane.

Although it is not intended that embodiments of this disclosure be limited by any particular mechanism, experiments were performed to examine the mechanism of NNA uptake into the cell and specifically the role of SRB1 for mediating uptake. SRB1 is reported to interact with the helices of ApoA1and ApoA1mimetic peptides bidirectional transfer of cargo into the cell using a non-endocytic mechanism. Leman et al. Journal of Medicinal Chemistry 2014, 57, 2169-2196. It is plausible that ASO conjugation to the peptide could interfere with the SRB1 recognition and disrupt ASO transfer, potentially diminishing the utility of the NNAs. SRB1 activity is diminished with a small molecule inhibitor named blocker of lipid transport (BLT-1). Nieland et al. PNAS, 2002, 99, 15422-15427. BLT-1 binds to the amino acid residue C384, which has a role in selective cellular uptake of SRB1. Yu et al. PNAS, 2011, 108, 12243-12248. Upon treating HeLa cells with BLT-1 (50 μM) and incubating with the panel of ASO-ND and NNA conjugates (1-5), cells were collected to assess the mean Cy5 fluorescence intensity of the ND scaffold (labeled with Cy5) via flow cytometry. Surprisingly, there was a significant reduction in the uptake of ASO-NDs and NNAs into cells following BLT-1 treatment for all the NDs tested. This reduction correlated into 56-73% reduction in uptake for all groups when the uptake was compared and normalized against the uptake for cells treated with ASO-NDs and NNAs without BLT-1 treatment. This partial reduction in uptake of ASO-NDs and NNAs suggest that greater than 50% of the uptake route involves other mechanisms. The covalent attachment of the ASO to phospholipids is not likely to not limit SRB1 uptake because phospholipids are also internalized by SRB1 through the selective lipid uptake pathway during cargo transport and catabolism of the HDL scaffold. Thuahnai et al., J Biol Chem, 2001, 276, 43801-43808. In contrast, the ND-forming peptide itself is not known to be trafficked using the SRB1 pathway, but it is possible that the amphipathic peptide-oligonucleotide conjugate may undergo internalization. While endocytosis may play a role for internalization for all nanoparticles, these experiments indicate SRB1-dependent uptake enhances the therapeutic potential of NNAs as it limits endosome entrapment and eventual degradation. These experiments indicate that the C-terminal Cys modified 22A peptide design and nucleic acid conjugation does retain the selective, non-endocytic features of the ND scaffold.

### Internalized ASO-NDs and NNAs Undergo Dissociation Within 24 H

To better characterize the integrity of ASO-NDs and NNAs after internalization into the cell, sensitized-FRET was used to determine lipid-nucleic acid proximity. HeLa cells were incubated with 100 nM of ASO-NDs (1-2) and NNA (3) for 4 and 24 h. After rinsing and nuclear staining of the cells, the samples were imaged on an epifluorescence microscope using a FRET cube equipped to measure FRET using the TYE563 and Cy5 wavelengths. FRET efficiency was determined by using a pixel-by-pixel analysis method that accounted for cross talk between the donor and acceptor channels. For all the ND sample types that were examined, a decrease in FRET efficiency was noticed when we compare values at 4 h and 24 h. This confirms that ASO-ND conjugates dissociate over this time window. There was no detectable FRET for the ND scaffold only, DNA only, and the ND mixed with the DNA at the 4 and 24 h time points. Because the donor and acceptor are not directly linked to the same molecule, the FRET efficiency for the ND conjugates is inherently lower due to a larger Förster radii. This is also true for the ASO-ND 2 where the ASO is conjugated to the scaffolded peptide. The FRET efficiencies measured for samples at t=4 h were 36% for ASO-ND 1, 24% for ASO-ND 2, and 48% for NNA 3. There was not sufficient uptake of ND at t=0 to quantify DNA-ND and NNA integrity at early time points. Thus, FRET efficiency was next measure at 24 h providing values of 23% for ASO-ND 1, 7% for ASO-ND 2, and 37% for NNA 3. This data indicates that the ASO-ND and NNA assemblies are gradually dissociating. The disassembly of ASO-ND constructs is contemplated to be due to the activity of a combination of proteases, lipases, and nucleases as well as the retro-Michael (maleimide-thiol) reaction which releases the ASO from the ND under physiological conditions in the cytoplasm (i.e., highly reducing environment from glutathione). However, it is not required that the ASO be strictly localized to the NNA for activity purposes. Release from the scaffold can enhance the activity of ASO in the cell to bind mRNA, recruit RNaseH, and block the ribosome.

### Quantifying NNA and ASO-ND Activity In Vitro

Experiments were performed to compare the activity of the ASO conjugated NDs and NNA scaffolds by measuring HIF-1-alpha transcript levels using qPCR. HeLa cells were incubated with 100 nM ASO-ND or NNA for 24 h before cells were lysed and RNA was collected for qPCR. All treatment groups had an ASO concentration of 100 nM. This was validated using the extinction of DNA at 260 nm. The NNA scaffolds showed activity towards reducing basal HIF-1-alpha mRNA levels. Of the constructs tested, ND 2 and ND 3 (NNA), which contain the C-terminal cystine having the amino acid sequence of PVLDLFRELLNELLEALKQKLKGGC (SEQ ID NO: 2), showed greater levels of activity compared to ND 4 and ND 5 (NNA), which contain the N-terminal cystine having the amino acid sequence of CGGPVLDLFRELLNELLEALKQKLK (SEQ ID NO: 3). Conjugation of the ASO to the C-terminal cysteine of the ApoA1 mimetic peptide generated NDs that were significantly more active than ASOs conjugated to the N-terminus of the peptide. Overall, NNA (ND 3) showed the greatest level of activity (58% reduction of cellular HIF-1-alpha levels), suggesting that creating a high density of ASO around the ND scaffold leads to improved activity per ASO. ASO-ND 4 showed the least activity (24% knockdown). This lower activity is further supported by TEM analysis and DLS that showed more broadly distributed NDs and a subpopulation of lipid assemblies with greater than 100 nm.

Because cancer cells typically have an overreliance on glycolysis (Warburg effect) and HIF-1-alpha expression maintains upregulated glycolysis levels, knockdown of HIF-1-alpha can contribute to reducing cell viability. The qPCR quantification of HIF-1-alpha was also validated by measuring cell viability of HeLa cells treated with 100 nM of ASO-NDs and NNAs for 24 h using an MTT assay. Overall, all ASO-ND and NNA conjugates used for this study displayed significant reduction in cell viability when compared against treatment with a scrambled ASO. Consistent with the qPCR results, NNA (ND 3) showed the greatest reduction in HeLa cell viability (42% reduction) compared to the other scaffolds (1-2, 4-5). Taken together, this data indicated NNAs using ND 3, as the most potent scaffold for gene regulation.

To validate the activity of NNAs in other cell types, HIF-1-alpha knockdown was tested in three other model cell lines including KPC, LX-2 human stellate, and HepG2. These cell lines were chosen because of their intrinsic overexpression of HIF-1-alpha and represent different disease models (e.g., KPC: pancreatic ductal adenocarcinoma, LX-2: hepatic fibrogenesis in NAFLD, and HepG2: hepatocellular carcinoma) which are often exacerbated by abnormal levels of hypoxia. In each of these cell lines, there was a significant decrease in cellular HIF-1-alpha levels when treated with 100 nM of NNA (ND 3) for 24 h compared to the scrambled ASO. The NNA treatment displayed slightly more activity compared to that of soluble ASO only. These data conclude that the NNA conjugate prepared from peptide B is active in vitro.

### NNAs Penetrate the Hypoxic Core of Tumor Spheroids and Display Activity

Tumors typically consist of a poorly oxygenated and poorly vascularized necrotic core. The highly hypoxic core presents with diffusional selectivity and some drugs face mass transport barriers which limits delivery to the necrotic core. Larger scaffolds and higher molecular weight drugs might experience a barrier to reaching the core of tumor spheroids. However, tumors and other malignant cell lines express SRB1 as a possible mechanism to deliver cargo into the spheroid. Experiments were performed to determine whether NNA can penetrate the hypoxic core of spheroids using the active NNA (ND 3) in experiments with the three-dimensional H1299 non-small cell lung cancer (NSCLC) spheroid model. Spheroids embedded in Matrigel™ were incubated with 100 nM of NNA or ASO for 24 h before visualizing using confocal microscopy. Images show that there was a marked increase in NNA uptake into the core of the spheroid compared to the ASO only treatment. The soluble ASOs tended to internalize into the quiescent and proliferating areas (closer to surface and edges) of the spheroid. This was quantified by running a radial profile analysis of n=14 spheroids. The radial scans were initiated from the center of the spheroid out to the edge as determined from the brightfield image. The radial profile of soluble ASOs showed weak signal in the core of the spheroid and a gradual increase before leveling off in the proliferating zone. Conversely, the NNA treatment shows a rather uniform level of ASOs throughout the spheroid, highlighting the deeper penetrative ability of these discs. This confocal analysis strongly suggests that NNAs would provide a more effective strategy to deliver greater doses of ASOs to tumors. To quantify total uptake, flow cytometry was used to compare ASO uptake when the ASO was linked to the NNA compared to soluble DNA. NNA uptake was significantly greater than that of the ASO only treatment when directly evaluated using flow cytometry. Thus, NNAs offer a significant improvement in delivery to tumor spheroids both in terms of total uptake as well as delivery to the hypoxic core.

Experiments were performed to evaluate the role of SRB1 in mediating NNA penetration to the necrotic core of spheroids. Spheroids were treated with 50 μM BLT-1 for 1 h before adding 6 nM Cy5-ND (150 nM ASO) for 2 h. Spheroids were dissociated into individual cells and the Cy5 fluorescence was measured using flow cytometry. There was a 54% decrease in uptake in the spheroids treated with BLT-1 compared to untreated spheroids. This confirms that SRB1 plays a role in NNA mediate delivery of ASOs to spheroids. It is contemplated that the small size of the ND, compared to other types of nanoparticles such as liposomes, expediates transport across the extracellular matrix and interstitial openings.

The activity of anti-HIF-1-alpha NNA was tested by treating the spheroids with 550 nM of ASO or NNA for 24 h. Treatment of spheroids at this dosage resulted in an average 49% reduction in cellular HIF-1-alpha levels. Additionally, spheroids treated with just ASO resulted in only a 16% reduction in cellular HIF-1-alpha mRNA. The NNA conjugate exhibited significant potency against the ASO only treatment, hence signifying the potential for using NNAs to deliver nucleic acids as a form of cancer therapy for mediating hypoxia and sensitizing malignant tumors for increased response from other drug treatments.

### Anti-HIF-1-Alph NNAs are Active In Vivo

Experiments were performed to determine whether NNAs are active in vivo using a mouse model. A single tail-vein injection of the NNA or ND scaffold was subsequently followed 48 h later with analysis of HIF-1-alpha gene expression in different tissues. EZN2968 anti-HIF-1-alpha ASO, TGGCAAGCATCCTGTA (SEQ ID NO: 5, bold TGG and TGT are LNA) was conjugated to the NNA, purified, and then quantified by UV-Vis to determine the concentration. NNA and ND solutions (200 μL of approximately 5 μM) that were doped with 1% Cy5 phospholipid were delivered. The DNA concentration was approximately 58 μM which is equivalent to a dose of 0.7 mg/kg of the ASO into C57BL/6 mice by tail-vein injection. Live trafficking of the NNA and ND scaffold was visualized in vivo using a whole-body imaging at t=6 h and 24 h post-injection. Most of the localization of the NNA at t=6 h was in the abdominal area (liver and kidneys), with some minor accumulation near the bronchial area. In contrast, the ND control scaffold was more uniformly distributed and was still present in the tail-vein at the 6 h time point. This localization of NNAs to the liver/kidney maybe the result of the ASO, which increases the molecular weight and hydrodynamic size of the particles, which also increases cell uptake. Within 24 h, the injected ND scaffold and NNA was localized primarily to the abdominal area. At 48 h post-injection, organs were harvested for ex vivo imaging. The NNA conjugates accumulated in liver, kidney, spleen, lung, and fat tissues which was also noted for the ND. The amount of uptake as inferred from the total fluorescence intensity of the tissues indicated comparable levels for the ND and NNA. Fluorescence quantification detailed that the kidney and liver were the two major organs for internalizing the NNA and ND scaffold. RNA was extracted from harvested organs and the relative HIF-1-alpha levels in each organ was evaluated through qPCR. Knockdown of HIF-1-alpha was note following NNA treatment in the liver and kidney tissues.

What is claimed is:

1. A phosphate membrane nanodisc covalently conjugated to a therapeutic agent, wherein the phosphate membrane nanodisc comprises
a zwitterionic phospholipid,
a nanodisc stabilizing peptide, and
wherein the therapeutic agent is conjugated to a phospholipid providing a thiol-linked adduct.

2. The phosphate membrane nanodisc of claim 1, wherein the therapeutic agent is a nucleobase polymer.

3. The phosphate membrane nanodisc of claim 2, wherein greater than 12 nucleobase polymers are conjugated to each phosphate membrane nanodisc and has a diameter of about between 10 and 20 nm.

4. The phosphate membrane nanodisc of claim 3 made by the process of contacting the zwitterionic phospholipid, a phospholipid having a thiol group, and a nanodisc stabilizing peptide, wherein the molar ratio of the zwitterionic phospholipid and the phospholipid having a thiol group is between 95 to 5 and 90 to 10.

5. The phosphate membrane nanodisc of claim 3 made by the process of contacting the zwitterionic phospholipid, a phospholipid having a thiol group, and a nanodisc stabilizing peptide, at a temperature between 40 and 55 degrees Celsius.

6. The phosphate membrane nanodisc of claim 3 made by the process of contacting the zwitterionic phospholipid, a phospholipid having a thiol group, and a nanodisc stabilizing peptide, in an aqueous solution at a pH between 7.5 and 8.5.

7. The phosphate membrane nanodisc of claim 1, wherein the zwitterionic phospholipid is 1,2-dimyristoyl-sn-glycero-3-phosphocholine (DMPC).

8. The phosphate membrane nanodisc of claim 4, wherein the phospholipid having a thiol group is 1,2-dipalmitoyl-sn-glycero-3-phosphothioethanol.

9. The phosphate membrane nanodisc of claim 8, wherein the molar ratio of the zwitterionic phospholipid to the phospholipid having a thiol group is at a ratio between 8:1 and 10:1.

10. The phosphate membrane nanodisc of claim 1, wherein the nanodisc stabilizing peptide comprises a C-terminal thiol group, cysteine amino acid, a GC sequence, or GGC sequence.

11. The phosphate membrane nanodisc of claim 10, wherein the nanodisc stabilizing peptide comprises the amino acid sequence of PVLDLFRELLNELLEALKQKLKGGC (SEQ ID NO: 2).

12. The phosphate membrane nanodisc of claim 1, wherein the therapeutic agent is a nucleobase polymer.

13. The phosphate membrane nanodisc of claim 12, wherein the nucleobase polymer comprises TGGCAAGCATCCTGTA (SEQ ID NO: 5).

14. A method of treating cancer comprising administering to a subject in need thereof an effective amount of a phosphate membrane nanodisc as provided for in claim 1 comprising a nucleobase polymer that specifically binds HIF-1-alpha mRNA.

15. The method of claim 14, wherein the nucleobase polymer comprises TGGCAAGCATCCTGTA (SEQ ID NO: 5).

16. The method of claim 14, wherein the cancer is pancreatic cancer, liver cancer, kidney cancer, lung cancer, non-small cell lung cancer, small cell lung cancer, or breast cancer.

17. A phosphate membrane nanodisc covalently conjugated to a therapeutic agent, wherein the nanodisc comprises
zwitterionic phospholipid,
a nanodisc stabilizing peptide comprising a C-terminal thiol group, C-terminal cysteine amino acid, a GC sequence, or GGC sequence,
wherein the therapeutic agent is conjugated to a phospholipid having a thiol-linked adduct; and
wherein the therapeutic agent is conjugated to the stabilizing peptide having a thiol-linked adduct to the C-terminal thiol group, C-terminal cysteine amino acid, a GC sequence, or GGC sequence.

18. The phosphate membrane nanodisc of claim 17, wherein the nanodisc stabilizing peptide comprises the amino acid sequence of PVLDLFRELLNELLEALKQKLKGGC (SEQ ID NO: 2).

19. The phosphate membrane nanodisc of claim 17, wherein the therapeutic agent is a nucleobase polymer comprising the sequence TGGCAAGCATCCTGTA (SEQ ID NO: 5).
